SEQUENCE LISTING (19 sequences)

SEQ ID NO: 1 - protein, 377 aa, homo sapiens
MAADLGPWNDTINGTWDGDELGYRCRFNEDFKYVLLPVSYGVVCVPGLCLNAVALYIFLCRLKTWNASTTYMFHLAVSDALYAASLPLLVYYYARGDHWPFSTVLCKLVRFLFYTNLYCSILFLTCISVHRCLGVLRPLRSLRWGRARYARRVAGAVWVLVLACQAPVLYFVTTSARGGRVTCHDTSAPELFSRFVAYSSVMLGLLFAVPFAVILVCYVLMARRLLKPAYGTSGGLPRAKRKSVRTIAVVLAVFALCFLPFHVTRTLYYSFRSLDLSCHTLNAINMAYKVTRPLASANSCLDPVLYFLAGQRLVRFARDAKPPTGPSPATPARRRLGLRRSDRTDMQRIEDVLGSSEDSRRTESTPAGSENTKDIRL

SEQ ID NO: 2 - protein, 595 aa, homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 3 - DNA, 22 bp, artificial
  RdRp SARSr-F
gtgaratggtcatgtgtggcgg

SEQ ID NO: 4 - DNA, 26 bp, artificial
  RdRp SARSr-R
caratgttaaasacactattagcata

SEQ ID NO: 5 - DNA, 25 bp, artificial
  RdRp SARSr-P2
caggtggaacctcatcaggagatgc

SEQ ID NO: 6 - DNA, 26 bp, artificial
  E Sarbeco P1
acactagccatccttactgcgcttcg

SEQ ID NO: 7 - DNA, 26 bp, artificial
  E Sarbeco F
acaggtacgttaatagttaatagcgt

SEQ ID NO: 8 - DNA, 22 bp, artificial
  E Sarbeco R
atattgcagcagtacgcacaca

SEQ ID NO: 9 - RNA, 19 nt, artificial
  siGENOME non targeting siRNA SMART POOL no 1
uagcgacuaaacacaucaa

SEQ ID NO: 10 - RNA, 19 nt, artificial
  siGENOME Non Targeting siRNA SMARTpool no 2
uaaggcuaugaagagauac

SEQ ID NO: 11 - RNA, 19 nt, artificial
  siGENOME Non Targeting  siRNA SMARTpool no 3
auguauuggccuguauuag

SEQ ID NO: 12 - RNA, 19 nt, artificial
  siGENOME Non Targeting  siRNA SMARTpool no 4
augaacgugaauugcucaa

SEQ ID NO: 13 - RNA, 19 nt, artificial
  siGENOME SMARTpool  anti-P2Y2 no 1
ugccuagggccaagcgcaa

SEQ ID NO: 14 - RNA, 19 nt, artificial
  siGENOME SMARTpool  anti-P2Y2 no 2
uaacuggagcuccgauuua

SEQ ID NO: 15 - RNA, 19 nt, artificial
  siGENOME SMARTpool  anti-P2Y2 no 3
ucucaggaguagucucaua

SEQ ID NO: 16 - RNA, 19 nt, artificial
  siGENOME SMARTpool  anti-P2Y2 no 4
agucaucguuuguguguau

SEQ ID NO: 17 - DNA, 29 bp, artificial
  pLKO.1 shP2Y2
atgttccacctggctgtgtctgatgcact

SEQ ID NO: 18 - DNA, 21 bp, artificial
  pLKO.1 sh-1Caspase-1
ataatgagagcaagacgtgtg

SEQ ID NO: 19 - DNA, 21 bp, artificial
  pLKO.1 sh-2Caspase-1
agcatcatcctcaaactcttc

(19) United States
(12) Patent Application Publication
(10) Pub. No.: US 2023/0302031 A1
(43) Pub. Date: Sep. 28, 2023

(54) Modulators Of Purinergic Receptors and Related Immune Checkpoint For Treating Acute Respiratory Distress Syndrome

(71) Applicant: Institut Gustave-Roussy, Villejuif (FR)

(72) Inventors: Jean-Luc PERFETTINI, Meaux (FR); Deborah LECUYER, Guyancourt (FR); Desiree TANNOUS, Paris (FR); Awatef ALLOUCH, Bry-sur-Marne (FR); Oliver DELELIS, Viroflay (FR); Frederic SUBRA, Chantilly (FR)

(73) Assignee: Institut Gustave-Roussy, Villejuif (FR)

(21) Appl. No.: 17/928,352

(22) PCT Filed: Jun. 1, 2021
(86) PCT No.: PCT/EP2021/064718

§ 371 Date: Nov. 29, 2022

(30) Foreign Application Priority Data
Jun. 2, 2020 (EP) 20305578.5
Oct. 27, 2020 (EP) 20306287.2

(51) Int. Cl. A61K 31/7048; A61K 45/06; A61P 11/00; A61P 31/14; A61K 31/196; A61K 31/64; A61K 31/191

(52) U.S. Cl. CPC A61K 31/7048; A61K 45/06; A61P 11/00; A61P 31/14; A61K 31/196; A61K 31/64; A61K 31/191

(57) ABSTRACT

The inventors herein show that purinergic receptors regulate the conversion of macrophage pro-inflammatory reprogramming into anti-inflammatory phenotype in patients suffering from COVID-19 disease. Moreover, they show that P2Y receptor agonists repress NLRP3 inflammasome-dependent IL-1b secretion, but also impair the replication and the cytopathogenic effects of SARS-CoV-2. These results therefore suggest that some purinergic receptors agonists can treat acute lung injury and respiratory disease that are associated with SARS-CoV-2 infection. In addition, their results show that antagonists of the purinergic receptors P2X impair the replication of said virus. The present invention therefore proposes to use purinergic receptors modulators and NLR3-P2Y2R immune checkpoint modulators to treat patients suffering from a virus-induced acute respiratory distress syndrome.

### SUMMARY OF THE INVENTION

The inventors herein show that purinergic receptors regulate the conversion of macrophage pro-inflammatory reprogramming into anti-inflammatory phenotype in patients suffering from COVID-19 disease. Moreover, they show that P2Y receptor agonists repress NLRP3 inflammasome-dependent IL-1b secretion, but also impair the replication and the cytopathogenic effects of SARS-CoV-2. These results therefore suggest that some purinergic receptors agonists can treat acute lung injury and respiratory disease that are associated with SARS-CoV-2 infection. In addition, their results show that antagonists of the purinergic receptors P2X impair the replication of said virus. The present invention therefore proposes to use purinergic receptors modulators and NLR3-P2Y2R immune checkpoint modulators to treat patients suffering from a virus-induced acute respiratory distress syndrome.

### BACKGROUND ART

coronavirus disease 2019 (COV1D-19) is a pandemic caused by a novel strain of β-coronavirus, severe acute respiratory syndrome-coronavirus 2 (SARS-CoV-2). While infection can be asymptomatic, especially in children and young adults, the most common symptoms of COVID-19 are fever and cough, with a majority of patients developing dyspnea, reflecting a tropism of the virus for the lung (Guan and Zhong, 2020). Most patients presenting with a mild disease develop an efficient immune response (Thevarajan et al., 2020) but some of them suddenly evolve into a severe form with acute respiratory distress syndrome needing admission in intensive care unit (ICU), indicative of critical condition, and often culminating into multi-organ dysfunction leading to death (Wang et al., 2020). SARS-CoV-2 belongs to the species coronavirus, in the genus Betacoronavirus and family Coronaviridae.

In humans, coronavirus infections can cause respiratory pathologies associated with symptoms similar to the common cold, bronchiolitis and more serious diseases such as the Severe Acute Respiratory Syndrome caused by SARS-CoV-1, which generated an epidemic in 2003, and the Middle Eastern Respiratory Syndrome caused by MERS-CoV, which generated an epidemic in 2012. SARS-CoV-2 is the Betacoronavirus causing the coronavirus epidemic of 2019-2020, generating the form of pneumonia known as coronavirus disease 2019 or COV1D-19.

On 18 Mar. 2020, there were more than 215,000 cases in 156 countries and 8,732 people had lost their lives (from Johns Hopkins CSSE and Dong et al., 2020). Despite the fact that disease severity (including oxygen saturation, respiratory rate, blood leukocyte/lymphocyte count and chest X-ray/CT manifestations) predicts poor clinical outcomes (Guan et al., 2020), physio-pathological mechanisms responsible for COVID-19 remain unknown.

Symptoms of infection with SARS-CoV-2 are roughly similar to those of seasonal influenza infections: they include fever, fatigue, dry cough, shortness of breath, difficult breathing, pneumonia, renal failure, and may lead to death in severe cases. The severity of clinical signs requires that approximately 20% of patients remain in hospital and 5% require admission to intensive care. The most serious forms are observed in people who are vulnerable because of their age (over 70) or associated diseases such as hypertension, diabetes and/or coronary heart disease.

It is of the most importance to identify therapeutic compounds for preventing and/or treating COVID-19 disease. Indeed, today, no specific therapy or vaccine is currently available for the prevention or treatment of SARS-CoV-2 viral infection. The majority of treatments currently received by COVID-19 patients are primarily aimed at alleviating the symptoms of fever, cough and dyspnea in order to promote spontaneous recovery.

Antiviral compounds, known for their therapeutic activity on other types of viruses, are currently tested in clinical trials. The French High Council for Public Health issued an opinion on Mar. 23, 2020 on the therapeutic recommendations for the management of COVID-19 disease. This official notice proposes in particular to use the following therapeutic compounds: Remdesivir, Lopinavir/Rotinavir and Hydroxychloroquine. Remdesivir has been initially developed for the treatment of Ebola virus infections. Remdesivir is a broad-spectrum antiviral compound, acting as a nucleoside analogue, specifically an adenosine analogue. Its presence misleads the viral polymerase and causes a reduction in viral RNA synthesis. Lopinavir is a viral protease inhibitor, previously used against the human immunodeficiency virus (HIV). Lopinavir inhibits the production of functional proteins by the new virions, thereby blocking the spread of the virus. Lopinavir is rapidly degraded in the body. For this reason, it is administered in fixed combination with Ritonavir, which inhibit cytochrome P450 monooxygenases, thereby slowing the degradation of Lopinavir by these enzymes. Hydroxychloroquine, initially known for its anti-malaria activity, has been shown to have an apparent efficacy in the treatment of Covid-19 (Yao et al., 2020). However, clinical data are still limited and controversial.

There is a pressing urgency to identify preventive and therapeutic treatments to impair the replication of the virus, or alleviate the symptoms of the COVID disease, in order to decrease its severity. In this context, identification of biological targets of the SARS-CoV-2 may allow to define novel therapeutic strategies.

Like SARS-CoV infection, SARS-CoV2 infection frequently leads to acute lung injury (ALI). Fatal acute respiratory disease (ARD) is the major complication in patients with severe disease (Wang et al., 2020). Most patients who died of ARD exhibited an acute onset of lung inflammation (Ware and Matthay, 2000; Herold et al., 2011), thus highlighting the urgent need to characterize molecular and cellular mechanisms responsible for virus-mediated lung inflammation.

The accumulation of pro-inflammatory macrophages in infected lungs has been extensively reported during SARS-CoV infection (Ware and Matthay, 2000; Herold et al., 2011) and was proposed as a key event during SARS-CoV pathology (Liu et al., 2019). Assuming that SARS-CoV-2 shares similar inflammatory responses with SARS-CoV, elucidating molecular mechanisms that drive the polarization of Lung macrophages towards pro-inflammatory could lead to the identification of novel targets for treatment of SARS-CoV2 mediated-ALI and ARD.

An increased secretion of the pro-inflammatory cytokines such as interleukin (IL)-1β, IFN-Inducible protein-10 (IP-10) IL-4 and IL-6 was detected during SARS-CoV-2 infection (Huang et al., 2020) and aberrant inflammatory CD14*CD16* monocytes were described in severe pulmonary syndrome patients (Zhou et al., 2020). The pro-inflammatory functional reprogramming of macrophages is dependent of Fc-gamma receptor signaling and may activate NLRP3-dependent signaling pathways (Liu et al., 2019, Duffy et al., 2016). Consistently, SARS-CoV open reading frame-8b was recently shown to trigger NLRP3 inflammasome activation (Shi et al., 2019¹⁵). Additionally, the transmembrane pore-forming viral Viroporin 3a (also known as SARS-COV 3a) was shown to activate the NLRP3 inflammasome in lipopolysaccharide (LPS)-primed macrophages (Chen et al., 2019¹⁶).

It has been reported that the (NLR) protein 3 NLRP3 interacts with the purinergic receptor P2Y2 and dictates the early steps of HIV-1 infection (Paoletti et al., 2019²⁰). More importantly, it was also shown that P2Y2 dictates NLRP3 degradation through ubiquitinylation (Paoletti et al., 2019²⁰), thus revealing that P2Y2 acts as negative regulator of NLRP3 activation.

Yet, despite all these results, the role of the NLRP3-P2Y2 immune checkpoint in the immune response during SARS-CoV infections has never been studied so far. There is a need to evaluate if the modulation of NLRP3-P2Y2 immune checkpoint and especially the purinergic receptor signaling pathways could represent an innovative therapeutic strategy to avoid SARS-CoV-2-induced ARD.

### DETAILED DESCRIPTION OF THE INVENTION

### Importance of Purinergic Receptors and Related Immune Checkpoint During the Covid Disease

Macrophages derived from monocyte precursors undergo specific differentiation depending on the local tissue environment. The various macrophage functions are linked to the type of receptor interaction on the macrophage and the presence of cytokines. Similar to the T helper type 1 and T helper type 2 (TH1-TH2) polarization, two distinct states of polarized activation for macrophages have been defined: the classically activated pro-inflammatory-macrophage phenotype and the alternatively activated anti-inflammatory macrophage phenotype. Similar to T cells, there are some activating macrophages and some suppressive macrophages. Therefore, macrophages should be defined based on their specific functional activities. Classically activated pro-inflammatory macrophages have the role of effector cells in TH1 cellular immune responses, whereas the alternatively activated (M2) macrophages appear to be involved in immunosuppression and tissue repair.

Granulocyte macrophage colony stimulating factor (GM-CSF) and macrophage colony stimulating factor (M-CSF) are involved in the differentiation of monocytes to these two categories of macrophages: on a one hand, human GM-CSF polarize monocytes towards the pro-inflammatory macrophage subtype with a “pro-inflammatory” cytokine profile (e.g. TNF-alpha, IL-1beta, IL-6, IL-12 and IL-23); on another hand, treatment of monocytes with M-CSF induces macrophages into producing “anti-inflammatory” cytokines (e.g. IL-10, TGF-beta and IL-1ra) characteristic of M2 macrophages.

LPS and the TH1 cytokine IFN-gamma polarize macrophages towards the pro-inflammatory phenotype which induces the macrophage to produce large amounts of IL-1beta, TNF, IL-12, and IL-23. This helps to drive antigen specific TH1 and TH17 cell inflammatory responses forward and thus participates to the clearance of invading microorganisms. The antimicrobial functions of pro-inflammatory macrophages are linked to up-regulation of enzymes, such as inducible nitric oxide synthase (iNOS) that generates nitric oxide from L-arginine. The secretion of IL-6, IL-23, and IL-1beta are important factors in the induction and maintenance of Th17 cells. In some cases inflammatory responses can trigger tissue damage (toxic activity or reactive oxygen), resulting in an uncontrolled macrophage inflammatory response which could become pathogenic. For example, uncontrolled macrophage inflammatory response participates in the pathogenesis of inflammatory bowel disease (IBD).

By contrast, exposure of macrophages to the TH2 cytokine IL-4 produces a M2 phenotype which induces the production of high levels of IL-10 and IL-1RA and low expression of IL-12. These cells reduce inflammation, are immunoregulators, promote tissue remodeling and tumor progression. Indeed, recent studies indicate that tumor-associated macrophages (TAMs) show an anti-inflammatory phenotype. These tumor-associated macrophages (TAM) produce interleukin IL-10 and transforming growth factor (TGF) β to suppress general antitumor immune responses. Meanwhile, TAMs promote tumor neo-angiogenesis by the secretion of pro-angiogenic factors and define the invasive microenvironment to facilitate tumor metastasis and dissemination. For these reasons, reducing the pool of anti-inflammatory TAMs has been considered as a relevant approach to anti-cancer therapy.

It has been previously reported that the NLR family member NLRP3 and the purinergic receptor P2Y2 physically interact (Paoletti et al., 2019²⁰), control macrophage functions (such as cytokine secretion, functional reprogramming and migration) and constitutes an innate immune checkpoint that acts on macrophages (WO 2016/185026). In this prior work, it was proposed to use P2Y2R inhibitors in order to enhance the pool of pro-inflammatory macrophages and treat viral infections. It was furthermore shown that P2Y2R agonists can reduce the macrophage pro-inflammatory polarization, reduce the secretion of inflammatory cytokines, increase the anti-inflammatory macrophages pool and therefore be useful in patients suffering from auto-immune diseases or inflammatory diseases.

The present inventors have now observed that P2Y2R agonists can surprisingly inhibit the hyper-inflammation that is detected in patients with COVID-19 and can also impair the replication of the virus. More precisely, they revealed that P2Y2 receptor agonists (such as UTP, Diquafosol and Denufosol) can reduce macrophage pro-inflammatory reprogramming, NLRP3 inflammasome activation and subsequent IL-1β secretion in response to IFNγ or LPS+ATP stimulation and can impair the replication and the cytopathogenic effects of SARS-CoV-2. They therefore propose to reprogram macrophage pro-inflammatory phenotype into anti-inflammatory phenotype through the modulation of NLRP3-P2Y2 immune checkpoint as a therapeutic option for treating patients with COVID-19.

The present inventors furthermore studied the effects of other modulators of purinergic receptors on the macrophage populations and on the inflammasome activation in COVID suffering patients.

They thereby observed that P2X receptor antagonists (such as pyridoxal phosphate-6-azophenyl-2′,4′-disutfonic acid (PPDAS)) can repress SARS-CoV2 viral replication, whereas the P2X7 receptor agonist 2′,3′-O-(4-benzoyl-benzoyl)ATP enhanced the viral replication. They therefore propose to use P2X receptor antagonists as a therapeutic option for treating patients with COVID-19.

Altogether, these results demonstrate that purinergic receptors and their ligands (the extracellular nucleotides) play a central role during SARS-CoV-2 infection. The present application provides the first evidence that therapeutic manipulation of these receptors represents a good opportunity for the treatment of COVID-19.

### Modulators of the Invention

In a first aspect, the present invention therefore proposes to modulate purinergic receptors and/or the related immune checkpoint (in particular the NLRP3-P2Y2 immune checkpoint) for treating subjects suffering from Acute Respiratory Disease Syndrome (ARDS). This modulation can be obtained by means of various “modulators”, which are explained below.

In a particular embodiment, said modulator can be a modulator of purinergic receptors. This modulation can be either direct or indirect. Direct modulation can be mediated by using agonists or antagonists of said receptors. Indirect modulation can be obtained by modifying the availability or the level of the ligands of these receptors, or by any means enabling to enhance or reduce the biological activity of these receptors indirectly.

Purinergic receptors, also known as purinoceptors, are a family of plasma membrane molecules that are found in almost all mammalian tissues. More specifically, they are involved in several cellular functions, including proliferation and migration of neural stem cells, vascular reactivity, apoptosis and cytokine secretion. The term purinergic receptor was originally introduced to illustrate specific classes of membrane receptors that mediate relaxation of gut smooth muscle as a response to the release of ATP (P2 receptors) or adenosine (P1 receptors). P2 receptors have further been divided into five subclasses: P2X, P2Y, P2Z, P2U, and P2T. To distinguish P2 receptors further, the subclasses have been divided into families of metabotropic (P2Y, P2U, and P2T) and ionotropic receptors (P2X and P2Z).

In a preferred embodiment, the modulator of the purinergic receptor is a direct modulator which is selected from: an agonist of a purinergic P2Y receptor and an antagonist of a purinergic P2X receptor.

P2Y receptors are a family of purinergic G protein-coupled receptors. They are activated by ATP, ADP, UTP, UDP and UDP-glucose. They are known to be widely distributed in the brain, heart, kidneys, and adipose tissue. To date, 8 P2Y receptors have been cloned in humans: P2Y1, P2Y2, P2Y4, P2Y6, P2Y11, P2Y12, P2Y13 and P2Y14. They display large-scale structural domains typical of GPCRs, consisting of seven hydrophobic transmembrane helices connected by three short extracellular loops and three variably sized intracellular loops; an extracellular N-terminus; and an intracellular C-terminus. The extracellular regions interact with the receptor ligands, while the intracellular regions activate the G protein, control receptor internalization, and mediate dimerization. Similar to other GPCRs, P2Y receptors can form both homodimers and heterodimers. These dimeric forms may vary significantly in their biochemical and pharmacological properties from the monomeric receptor. In addition to the structural domains typical of all GPCRs, some structural elements are common across P2Y receptor subtypes. All P2Y receptors contain four extracellular cysteine residues which can form two disulfide bridges, one between the N-terminus domain and the proximal extracellular loop and another between the two remaining extracellular loops. These disulfide bonds have been shown to be involved in ligand binding and signal transduction. In addition, several polar residues found within the transmembrane helices are highly conserved across both species and receptor subtypes. Mutational analysis has suggested that these residues are integral to the ligand-binding mechanism of P2Y receptors. Outside of these conserved regions, the P2Y receptor family exhibits unusually high diversity in primary structure, with P2Y1 sharing only 19% of its primary structure with P2Y12. Despite this, the individual P2Y subtypes are highly conserved across species, with human and mouse P2Y receptors sharing 95% of amino acids.

P2Y receptors respond either positively or negatively to the presence of nucleotides in extracellular solution. Nucleotides may be divided into two categories: purines and pyrimidines. Some P2Y receptor species may respond to only purines, only pyrimidines, or both; for example, P2Y1 respond only to purines, P2Y4 respond only to pyrimidine, P2Y14 is activated only by UDP-glucose, and P2Y2 is activated by both purines and pyrimidines triphosphate (Tulapurkar et al., 2005).

The activity of P2Y receptors is linked to a signal cascade originating in regulation of the flow of Ca2+ and K+ ions by the receptor's interactions with G proteins, modulating access to Ca2+ and K+ channels. Voltage-independent Ca2+ channels allow for the free flow of Ca2+ ions from the cell activated by P2Y receptors (Van Bolen et al., 2006).

By contrast, P2X receptors are ligand-gated ion channels. These ligand-gated ion channels are nonselective cation channels responsible for mediating excitatory postsynaptic responses, similar to nicotinic and ionotropic glutamate receptors. P2X receptors are distinct from the rest of the widely known ligand-gated ion channels, as the genetic encoding of these particular channels indicates the presence of only two transmembrane domains within the channels. These receptors are greatly distributed in neurons and glial cells throughout the central and peripheral nervous systems. P2X receptors mediate a large variety of responses including fast transmission at central synapses, contraction of smooth muscle cells, platelet aggregation, macrophage activation, and apoptosis (North R A et al, 2002).

P2X receptors are expressed in cells from a wide variety of animal tissues. P2X receptors are able to initiate contraction in cells of the heart muscle, skeletal muscle, and various smooth muscle tissues, including that of the vasculature, vas deferens and urinary bladder. P2X receptors are also expressed on leukocytes, including lymphocytes and macrophages, and are present on blood platelets. There is some degree of subtype specificity as to which P2X receptor subtypes are expressed on specific cell types, with P2X1 receptors being particularly prominent in smooth muscle cells, and P2X2 being widespread throughout the autonomic nervous system.

However, such trends are very general and there is considerable overlap in subunit distribution, with most cell types expressing more than one subunits. For example, P2X2 and P2X3 subunits are commonly found co-expressed in sensory neurons, where they often co-assemble into functional P2X2/3 receptors.

Generally speaking, most subunits can form functional homomeric or heteromeric receptors. Receptor nomenclature dictates that naming is determined by the constituent subunits; e.g. a homomeric P2X receptor made up of only P2X1 subunits is called a P2X1 receptor, and a heteromeric receptor containing P2X2 and P2X3 subunits is called a P2X2/3 receptor. The general consensus is that P2X6 cannot form a functional homomeric receptor and that P2X7 cannot form a functional heteromeric receptor. Topologically, they resemble the epithelial Na+ channel proteins in possessing (a) N- and C-termini localized intracellularly, (b) two putative transmembrane segments, (c) a large extracellular loop domain, and (d) many conserved extracellular cysteyl residues. P2X receptor channels transport small monovalent cations, although some also transport Ca²⁺. Evidence has strongly indicated that the functional P2X receptor protein is a trimer, with the three peptide subunits arranged around an ion-permeable channel pore (Kawate et al., 2009). These findings indicate that the second transmembrane domain of each subunit lines the ion-conducting pore and is therefore responsible for channel gating.

The P2X receptors open in response to the binding of extracellular adenosine 5′ triphosphate (ATP). Three ATP molecules are thought to be required to activate a P2X receptor, suggesting that ATP needs to bind to each of the three subunits in order to open the channel pore, though recent evidence suggests that ATP binds at the three subunit interfaces. Once ATP binds to the extracellular loop of the P2X receptor, it evokes a conformational change in the structure of the ion channel that results in the opening of the ion-permeable pore. The most commonly accepted theory of channel opening involves the rotation and separation of the second transmembrane domain (TM) helices, allowing cations such as Na⁺ and Ca²⁺ to access the ion-conducting pore through three lateral fenestrations above the TM domains. The entry of cations leads to the depolarization of the cell membrane and the activation of various Ca²⁺ sensitive intracellular processes,

As used herein, the term “P2Y2 receptor” has its general meaning in the art and refers to the P2Y purinoreceptor 2 also known as P2RY2, HP2U, P2RU1, P2U, P2U1, P2UR, P2Y2, P2Y2R, and purinergic receptor P2Y2. This receptor protein of 377 amino acids is a G-protein-coupled receptor with seven transmembrane-spanning domains. It is referenced in public available bases as NP_002555 (SEQ. ID NO:1). Said receptor protein is encoded by the P2RY2 gene in humans. Three human transcript variants encode the same 377 amino acid protein sequence: NM_002564, NM_176071, NM_176072.

As used herein, the term “P2X7 receptor” has its general meaning in the art and refers to the P2X purinoreceptor 7 known depicted as NP_002553 (SEQ ID NO:2). It is a 595-amino acid polypeptide with two membrane-spanning domains. Said receptor is encoded by the P2X7 gene in humans, for example by the mRNA NM_002562.

In another particular embodiment of the invention, the modulator of the invention is able to modulate the NLRP3-P2Y2 immune checkpoint. In this case, said modulator is for example able to impair the activity of the NLRP3 inflammasome.

The NLRP3 inflammasome, so-called because the NLRP3 protein in the complex belongs to the family of nucleotide-binding and oligomerization domain-like receptors (NLRs), is also known as “pyrin domain-containing protein 3”. NLRP3 assembles a multimeric inflammasome complex serving as an activation platform for caspase-1 that controls processing and release of cytosolic inflammatory factors and cytokines including IL-1β. Inflammasome assembly is tightly controlled and requires coordinated NLRP3 priming, through cytokine or other pattern recognition receptors, followed by activation by cellular stress. The present inventors demonstrated that it is involved in the pro-inflammatory response associated with virus-induced ARDS, notably due to SARS-CoV2.

As used herein, the term “NLRP3” has its general meaning in the art and refers to the “NACHT, LRR and PYD domains-containing protein 3”. An exemplary human amino acid sequence is represented by NP_04886 (isoform a of 1036 amino acids).

Several NLRP3 inflammasome inhibitors have been described, some of which show promise in the clinic (see below and in Shao et al, 2015).

To reduce the inflammasome activity, it is also possible to use compounds that act as antagonists of NRLP3. These compounds are for example Troxerutin, Tranilast or Glyburide.

It is possible to detect the inflammasome activity by common molecular means (such as CaspaseGlo® of Promega, detection of IL-1b secretion using ELISA or Western blot or NLRP3 inflammasome formation using Fluorescence Resonance Energy Transfer (FRET) Assay).

### Compounds of the Invention

As used herein, the term “P2Y receptor agonist” refers to any compound that enhances the biological activity of at least one of the P2Y receptors as defined above (P2Y1, P2Y2, P2Y4, P2Y6, P2Y11, P2Y12, P2Y13 and P2Y14).

In a preferred embodiment, said P2Y receptor agonist of the invention is able to enhance the activity of the P2Y2 receptor. In the presence of this agonist, the P2Y2 receptor will perform its biochemical or its cellular function with enhanced efficiency. For example, such an agonist can act by making the receptor more accessible to its natural ligand (e.g. ATP) so that its normal biological activity is enhanced. The agonistic activity of compounds towards the P2Y2 receptors may be determined using various methods well known in the art. For example, the assay can be performed with P2Y2 receptor expressed on the surface of cells. A typical assay for determining the agonistic activities of a compound on P2Y2 receptor is described in Hillmann et al.,2009 and in Van Poecke et al, 2012.

By “agonist”, it is herein meant either a small chemical molecule or a larger protein (such as antibody) that interact physically with the target receptor so as to enhance its biological activity, e.g., by rendering the receptor more accessible to its ligand.

Chemical P2Y2 receptor agonists are well known in the art and include those described in U.S. Pat. Nos. 5,789,391, 5,837,861, and US 2002/0082417. Suitable chemical P2Y2R agonists typically include INS-37217 [P(1)-(uridine 5′)-P(4)-(2′-deoxycytidine 5′)tetraphosphate tetrasodium salt], uridine 5′triphosphate, diquafosol tetrasodium, and the like. In some embodiments, P2Y2 receptor agonists are selected from the compounds described in WO 2008/060632, which is incorporated herein by reference. It is also possible to use activating antibodies. With this respect, monoclonal antibodies can be produced and screened for their capacity to enhance the activity of the P2Y2 receptor.

In a more preferred embodiment, the P2Y receptor agonist of the invention is a small chemical compound selected in the group consisting of: MRS2698, Uridine triphosphate (UTP), 4-thio-UTP, 2-thioUTP, Diquafosol, PSB1114, ATP, Denufosol, Ap4A, UTPγS, 5BrUTP and MR52768 and any pharmaceutically acceptable salt thereof (Jacobson et al, 2009; 2012).

As used herein, the term “P2X receptor antagonist” refers to any compound that impairs or blocks or reduces the biological activity of at least one of the P2X receptors as defined above (P2X1, P2X2, P2X3, P2X4, P2X5, P2X6, P2X7). The antagonistic activity of compounds towards the P2X receptors may be determined using various methods well known in the art. For example, the agents may be tested for their capacity to block the interaction of P2X receptor with its natural ligand receptor (e.g. periodate-oxidized ATP), or to reduce the biological activity of the P2X receptor without impairing the binding of the ligand.

By “antagonist”, it is herein meant either a small chemical molecule that can directly interact with the target receptor, or polypeptides (such as antibodies or aptamers) that can block the interaction between the target receptor and its ligand. More generally, it can also encompass gene expression inhibitors (siRNAs, ribozymes, etc) that can inhibit the production of the protein in target cells.

Chemical P2X receptor antagonists are for example NF279 (P2X1 antagonist), NF449 (P2X1 antagonist), Suramin (P2X1 and P2X5 antagonist), TNP-ATP (P2X1 and P2X4 antagonist), Ip5I (P2X1 antagonist) and NF023 (P2X1 antagonist), NF778 (P2X2 antagonist), NF770 (P2X2 antagonist), A317491 (P2X3 antagonist), gefapixant (P2X3 antagonist), PSB-12062 (P2X4 antagonist), 5-BDBD (P2X4 antagonist) and any pharmaceutically acceptable salt thereof (North R. A. and Jarvis M. F., 2013). It is also possible to use antibodies/aptamers that target the binding site of P2X receptor in order to impair the binding of its ligand.

In a preferred embodiment, the P2X receptor antagonist of the invention is able to impair the activity of the P2X7 receptor.

In a preferred embodiment, the P2X receptor antagonist of the invention is able to impair the activity of the P2X7 receptor and is chosen in the group consisting of: JNJ-47965567 (Bhattacharya et al, 2013), Compound 16i (Homerin et al, 2019), AZ10606120 (Guile et al 2009; Michel et al, 2008), AZD9056 (McInnes et al,2014), GSK1482160 (Abdi et al, 2010; Homerin et al, 2019), A438079 (Donnelly-and Jarvis, 2007; Khalafalla et al.,2017), BBG (Carmo et al., 2014), KN62 (Gargett and Wiley, 1997), OxATP (Lowe and Beechey 1982), A71003 [N-(1-{[(cyanoimino)(5-quinolinylamino)methyl]amino}-2,2-dimethylpropyl)-2-(3,4-dimethoxyphenyl)acetamide] (Honore et al, 2006), AACBA [N-(adamantan-1-ylmethyl)-5-[(3R-amino-pyrrolidin-1-yl)methyl]-2-chloro-benzamide] (Broom et al., 2008) and CE-224,535 (or PF-04905428) (Duplantier A J, et al 2011) and any pharmaceutically acceptable salt thereof.

It is also possible to use antibodies/aptamers that target the binding site of P2X7 receptor in order to impair the binding of its ligand. In this embodiment, the inhibitor of P2X receptor may consist in an antibody (this term including antibody fragment). In particular, it can be an antibody directed against the P2X receptor in such a way that said antibody impairs the activation of said receptor. Antibodies can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies can be prepared and isolated using any technique that provides for the production of antibody molecules by continuous cell tines in culture. Techniques for production and isolation include but are not limited to the hybridoma technique; the human B-cell hybridoma technique; and the EBV-hybridoma technique. Alternatively, techniques described for the production of single chain antibodies (see, e.g., U.S. Pat. No. 4,946,778) can be adapted to produce anti-P2X receptor, single chain antibodies. The inhibitor of P2X receptor activity of the invention also include anti-P2X receptor antibody fragments including but not limited to F(ab′)2 fragments, which can be generated by pepsin digestion of an intact antibody molecule, and Fab fragments, which can be generated by reducing the disulfide bridges of the F(ab′)2 fragments.

Alternatively, Fab and/or scFv expression libraries can be constructed to allow rapid identification of fragments having the desired specificity to the receptor or channel. Humanized antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397). In view of these information, the skilled in the art can easily generate and select antibodies blocking the P2X receptor.

The inhibitor of P2X can also be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods. In view of these information, the skilled in the art can easily generate and select aptamers blocking the P2X receptor.

In the context of the invention, it is also possible to use inhibitors of P2X receptor expression, that will efficiently reduce or abolish the activity of the P2X receptor. Such inhibitor can be used so that (i) the transcription of the gene encoding P2X receptor is lowered, i.e. the level of its mRNA is lowered or (ii) the translation of the mRNA encoding P2X receptor is lowered.

The P2X modulator of the invention can also inhibit the P2X receptor gene expression. It is for example a natural or synthetic compound that has a biological effect to inhibit or significantly reduce the expression of a P2X gene, e.g., of the P2X7R gene. Inhibitors of gene expression for use in the present invention may be based on anti-sense oligonucleotide constructs. Anti-sense oligonucleotides, including anti-sense RNA molecules and anti-sense DNA molecules, would act to directly block the translation of the mRNA by binding thereto and thus preventing protein translation or increasing mRNA degradation, thus decreasing the level of the protein (e.g. P2X receptor), and thus activity, in a cell. For example, antisense oligonucleotides of at least about bases and complementary to unique regions of the mRNA transcript sequence encoding the targeted protein (e.g. P2X receptor) can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of gene expression for use in the present invention. Gene expression can be reduced by contacting a subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschl et al., 1999; Elbashir et al., 2001; Hannon, 2002; McManus et al., 2002; Brummelkamp et al., 2002; U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone. The antisense oligonucleotides siRNAs and ribozymes of the invention may be delivered in vivo alone or in association with a vector. Preferred vectors are defined below.

It is also possible to modulate indirectly the activity of purinergic receptors by modifying the availability and/or the quantity of their ligand (e.g., ATP) in the surroundings of the macrophage cells.

In another embodiment, the modulator used in the invention is therefore able to reduce the level of circulating extracellular nucleotides such as ATP. To do so, it is possible to use, e.g., the human recombinant apyrase AZD3366 (ATP102) (Moeckel et al., 2014) or probenecid.

In the examples below, the inventors have shown that the use of P2Y2 agonists trigger the inactivation of the NLRP3 inflammasome in activated macrophages. It is therefore also possible to partially mimic the effect of the P2Y2 agonists by using NLRP3 inflammasome inhibitors such as MCC950 (Coll et al., 2015) or beta-Hydroxybutyrate (Yawn et al., 2015), or NLR3P antagonists such as Troxerutin (Sun et al., 2016), Tranilast (Huang et al., 2018) and Glyburide (Lamkanfi et al., 2009).

It is also possible to use, as for P2X7R, any inhibitor that affect the expression level of NLRP3 (siRNAs, ribozymes, etc) or any antibody/aptamer that block the activity of the NLRP3 protein.

The term “P2Y2R modulator” therefore also encompasses, in the context of the invention, NLRP3 antagonists such as Troxerutin, Tranilast and Glyburide, and NLR3 inflammasome inhibitors such as MCC950 beta-Hydroxybutyrate.

By “modulators of the invention” or “compounds of the invention” it is therefore herein encompassed:
A modulator of purinergic receptors, preferably an agonist of a purinergic P2Y receptor or an antagonist of a purinergic P2X receptor, more preferably an agonist of the purinergic P2Y2 receptor or an antagonist of the purinergic P2X7 receptor, as defined above,
A modulator that impair the activity of the NRLP3 inflammasome, e.g., an antagonist of NLR3P or an NLRP3 inflammasome inhibitor, as defined above, and
A modulator that reduce the level of extracellular nucleotides such as ATP, as defined above.

### Therapeutic Uses of These Modulators

The examples below suggest that the modulation of NLRP3-P2Y2 immune checkpoint and other purinergic receptor signaling pathways represent an innovative therapeutic strategy to avoid SARS-CoV-2-induced Acute Respiratory Distress Syndrome (ARDS).

The present invention therefore relates on the use of the modulators of the invention for treating subjects suffering from an acute respiratory distress syndrome ARDS.

The present invention also encompasses the use of any of the compound of the invention for manufacturing a pharmaceutical composition intended to treat subjects suffering from ARSD. In these pharmaceutical compositions, the compounds of the present invention (e.g. P2Y2 receptor agonist or the inhibitor of P2X receptor activity) are typically combined with pharmaceutically acceptable excipients or sustained-release matrices (such as biodegradable polymers). The present invention also relates to treatment methods comprising the step of administering a therapeutically effective amount of the modulators/compounds of the invention to subjects in need thereof.

Acute respiratory distress syndrome (ARDS) is a type of respiratoryfailure characterized by rapid onset of widespread inflammation in the lungs (Fan et al, 2018). Symptoms include shortness of breath, rapid breathing, and bluish skin coloration. The underlying mechanism involves diffuse injury to cells which form the barrier of the microscopic air sacs of the lungs, surfactant dysfunction, activation of the immune system, and dysfunction of the body's regulation of blood clotting (Fanelli et al., 2015). ARDS impairs the lungs' ability to exchange oxygen and carbon dioxide. Adult diagnosis is based on a PaO₂/FiO₂ ratio (ratio of partial pressure arterial oxygen and fraction of inspired oxygen) of less than 300 mm Hg despite a positive end-expiratory pressure (PEEP) of more than 5 cm H₂O. The primary treatment involves mechanical ventilation together with treatments directed at the underlying cause. Ventilation strategies include using low volumes and low pressures. If oxygenation remains insufficient, lung recruitment agents and neuromuscular blockers may be used. If these are insufficient, extracorporeal membrane oxygenation (ECMO) may be an option. The syndrome is associated with a death rate between 35 and 50% (Fan et al, 2018).

The acute respiratory distress syndrome (ARDS) can be induced by various causes, as shown in Table 1 below, which is an abbreviated list of the common causes of ARDS.

| Etiology | Clinical features | Diagnostic tests |
|---|---|---|
| Sepsis | Fever hypotension, leukocytosis, lactic | Appropriate clinical context and |
| | acidosis, infectious source | positive cultures |
| Aspiration pneumonitis | Witnessed or risk for aspiration, food, lipid laden | Presumptive diagnosis with negative |
| | macrophages, airway erythema on bronchoscopy | cultures |
| Infectious pneumonia (including mycobacterial, | Productive cough, pleuritic pain, fever, leukocytosis, | Appropriate clinical context and |
| viral, fungal, parasitic) | lobar consolidation or bilateral infiltrates | positive respiratory cultures |
| | in an immunosuppressed patient | |
| Severe trauma and/or multiple fractures | History of trauma of fractures within the last week | Diagnosis is apparent |
| Pulmonary contusion | History of chest trauma (blunt or penetrating), | Presumptive diagnosis in the correct |
| | chest pain | clinical context, negative cultures |
| Burns and smoke inhalation | Exposure to fire or smoke, cough, dyspnea, DIC, | Presumptive diagnosis in the correct |
| | particulate matter on bronchoscopy, surface burns | clinical context, negative cultures |
| Transfusion related acute lung | History of transfusion, dyspnea during or shortly | Diagnosis of exclusion |
| injury and massive transfusions | after transfusion | |
| HSCT^{¶} | History of HSCT | Diagnosis of exclusion |
| Pancreatitis | Abdominal pain, vomiting, risk actors (eg, gallstones, | Elevated amylase and lipase, with or |
| | alcohol, viral infection) | without abnormal imaging |
| Inhalation injures other than | History of inhalation exposure (eg, chlorine gas) | Diagnosis of exclusion |
| smoke (eg, near drowning, gases) | | |
| Thoracic surgery (eg, post-cardiopulmonary | History of surgery, intraoperative ventilation, | Diagnosis of exclusion |
| bypass) or other major surgery | intraoperative transfusion | |
| Drugs (chemotherapeutic agents, | New drugs or radiation exposure on history, | Diagnosis of exclusion, lung biopsy |
| amiodarone, radiation) | lymphocytosis on lavage, lavage may have | occasionally helpful |
| | suggestive features of amiodarone toxicity | |
| | (“foamy macrophages”) but is nonspecific | |

Table 1. Etiology of ARDS: acute respiratory distress syndrome; DIC: disseminated intravascular coagulation; HSCT: hematopoietic stem cell transplant; AEP: acute eosinophilic pneumonia; COP: cryptogenic organizing pneumonia; DAD: diffuse alveolar.

It is possible to use the modulators of the invention to treat ARDS whatever its etiology is. In particular, it is possible to use the modulators of the invention to treat ARDS caused by sepsis, pneumonia, pancreatitis, surgery, radiation or chemotherapeutic drugs, etc.

ARDS is a clinical diagnosis of exclusion: it can only be diagnosed once cardiogenic pulmonary edema and alternative causes of acute hypoxemic respiratory failure and bilateral infiltrates have been excluded. The Berlin Definition of ARDS requires that all of the following criteria be present for diagnosis:
Respiratory symptoms must have begun within one week of a known clinical insult, or the patient must have new or worsening symptoms during the past week.
Bilateral opacities must be present on a chest radiograph or computed tomographic (CT) scan. These opacities must not be fully explained by pleural effusions, lobar collapse, lung collapse, or pulmonary nodules.
The patient's respiratory failure must not be fully explained by cardiac failure or fluid overload. An objective assessment (e.g., echocardiography) to exclude hydrostatic pulmonary edema is required if no risk factors for ARDS are present.
A moderate to severe impairment of oxygenation must be present, as defined by the ratio of arterial oxygen tension to fraction of inspired oxygen (PaO₂/FiO₂).

The severity of the hypoxemia defines the severity of the ARDS:
Mild ARDS—The PaO₂/FiO₂ is >200 mmHg, but ≤300 mmHg, on ventilator settings that include positive end-expiratory pressure (PEEP) or continuous positive airway pressure (CPAP)≥5 cm H₂O.
Moderate ARDS—The PaO₂/FiO₂ is >100 mmHg, but ≤200 mmHg, on ventilator settings that include PEEP≥5 cm H₂O.
Severe ARDS—The PaO₂/FiO₂ is ≤100 mmHg on ventilator settings that include PEEP≥5 cm H₂O.

Determining the PaO₂/FiO₂ requires arterial blood gas (ABG) analysis. To calculate the PaO₂/FiO₂ ratio, the PaO₂ is measured in mmHg and the FiO₂ is expressed as a decimal between 0.21 and 1. As an example, if a patient has a PaO2 of 60 mmHg while receiving 80 percent oxygen, then the PaO₂/FiO₂ ratio is 75 mmHg (ie, 60 mmHg/0.8). For patients in whom an ABG cannot be obtained, the ratio of oxyhemoglobin saturation measured by pulse oximetry (SpO2) to FiO2 may be an appropriate substitute.

In addition, ARDS can be induced by viruses. Two virus types have been involved in the aetiology of this disease: respiratory viruses that cause community-acquired viral pneumonia and Herpesviridae that cause nosocomial viral pneumonia (Luyt et al., 2011). Among the respiratory viruses that can affect the lung and cause ARDS, pandemic viruses head the list, with influenza viruses H5N1 and H1N1 2009 being recently identified. Other viruses can cause severe ARDS. Notably, novel coronaviruses have been responsible for the severe acute respiratory syndrome outbreaks in 2003 and in 2019.

In a particular embodiment, the present invention relates on the use of the modulators of the invention for treating subjects suffering from a virus-induced acute respiratory distress syndrome ARDS.

In a preferred embodiment, the modulators of the invention are thus administered to subjects suffering from an ARDS caused by an influenza virus (such as H1N1 or H5N1), a respiratory virus, or a herpesvirus. All these viruses have indeed been shown to cause ARDS in infected humans (Luyt et al., 2011).

The term “herpesvirus” (or “Herpesviridae”) herein designates any herpesvirus that has been shown to induce an ARDS in an animal. It can be for example the Herpes simplex virus (HSV) or the Cytomegalovirus (CMV) (Luyt et al., 2011).

The term “respiratory virus” herein encompasses parainfluenza viruses, adenoviruses, respiratory syncytial viruses, coronaviruses and the metapneumovirus.

coronaviruses are enveloped viruses with a helically symmetrical capsid. They have a single-stranded, positive-sense RNA genome and are capable of infecting cells in birds and mammals. The morphology of the virions is typical, with a halo of protein protuberances (Spike) which gave them their name of ‘crown virus’. Four genera have been identified: Alphacoronavirus, Betacoronavirus, Gammacoronavirus, Deltacoronavirus. They can all infect humans.

In one embodiment, the modulators of the invention are administered to subjects that have been infected by at least one coronavirus.

In a particular embodiment, the modulators of the invention are administered to subjects that have been infected by at least one Betacoronavirus.

The Betacoronavirus genus (β-CoVs or Beta-CoVs), comprising virus infecting animals and/or humans, is subdivided into four lineages designated as A, B, C and D: Lineage A also designated as subgenus Embecovirus includes HCoV-OC43 and HCoV-HKU1, virus able to infect various species; Lineage B also designated as subgenus Sorbecovirus includes SARS-CoV-1, SARS-CoV-2, and Bat SL-COV-WIV1; Lineage C also designated as subgenus Merbecovirus includes Tylonycteris bat coronavirus HKU4 (BtCoV-HKU4), Pipistrellus bat coronavirus HKU5 (BtCoV-HKU5), and MERS-CoV, able to infect notably camels and humans; Lineage D also designated as subgenus Nobecovirus includes Rousettus bat coronavirus HKU9 (BtCoV-HKU9).

As used herein, the term “Betacoronavirus” designates any virus belonging to the Betacoronavirus genus (β-CoVs or Beta-CeVs) within the Coronaviridae family, in particular any Betacoronavirus belonging to one of the four lineages designated as A, B, C and D. It designates a Betacoronavirus infecting animals (preferably a mammal) and/or humans. In particular, this designation includes the Betacoronaviruses infecting human organisms selected from the group consisting of OC43, HKU1, SARS-CoV-1, SARS-CoV-2 and MERS-CoV.

The Betacoronaviruses of the greatest clinical importance concerning humans are:
OC43 and HKU1 of the A lineage,
SARS-CoV-1 and SARS-CoV-2 of the B Lineage, and
MERS-CoV of the C lineage.

In a more preferred embodiment, the modulators of the invention are administered to subjects that have been infected by a Betacoronavirus (such as OC43, HKU1, MERS-CoV, SARS-CoV-1 and SARS-CoV-2). In this preferred embodiment, the subjects treated by the invention are said to suffer from a COVID disease. As used herein, the terms “COM disease” or “COVID” or “Betacoronavirus disease” mean the disease linked to (associated with) the infection with at least one Betacoronavirus, as listed above.

In an even more preferred embodiment, the modulators of the invention are administered to subjects that have been infected by the SARS-CoV-2 virus. “SARS-CoV-2” herein refers to Severe Acute Respiratory Syndrome coronavirus 2. SARS-CoV-2 belongs to the species coronavirus, in the genus Betacoronavirus and family Coronaviridae. In this embodiment, the subjects treated by the invention are said to suffer from a COVID 19 disease. As used herein, the terms “COVID-19 disease” or “COVID-19” or “coronavirus disease 19” indeed mean the disease linked to (or associated with) the infection with (at least) the SARS-CoV-2 (Severe Acute Respiratory Syndrome coronavirus 2).

Viral diseases can manifest several forms, depending on the severity of the symptoms/signs. They can be asymptomatic in some people. They can induce a simple fever accompanied by cough in others. The early COVID symptoms (such as COVID-19) comprise: dry cough, muscle pain, headache, fever, fatigue, loss of taste or smell. They can ultimately cause acute respiratory distress and death.

In particular, for the Betacoronavirus disease (such as COVID-19), the following forms are usually observed:
an “asymptomatic form” wherein the subject is a carrier of at least on Betacoronavirus but shows no symptom.
a “mild COVID form” wherein the subject shows the first symptoms (or signs) of a COVID disease, as listed above. Mild COVID symptoms (such as COVID-19) comprise e.g., mild dry cough, mild muscle pain, mild headache, mild fever, mild fatigue, loss of taste or smell.
a “strong COVID form” wherein the subject shows strong respiratory symptoms such as difficulty breathing, lack of oxygen; other stronger symptoms such as fever, dry cough, aches and pains, nasal congestion, strong headache, conjunctivitis, sore throat, skin rash, discoloration of fingers or feet, or any combination thereof; as well as a deterioration of the general state of health with frequent diarrhoea, but also liver or urinary disorders, dizziness or neuromuscular problems; some of these symptoms may require hospitalisation. Most patients have an abnormal chest X-ray or CT scan within the first few days of illness, even in the absence of respiratory signs.
A “severe COVID form” or “critical COVID form” or “aggressive COVID form” wherein the subject has life-threatening symptoms (or signs) of COVID (e.g. COVID-19), comprising at least one selected from (but not limited to): respiratory distress, lung disorders, liver disorders, kidney disorders, neuromuscular disorders, brain disorders, etc, that requires hospitalisation and/or intensive care (in intensive care units (ICU)).

More generally, it is possible to use the modulators of the invention to treat inflammatory diseases that are associated with an accumulation of pro-inflammatory macrophages and/or associated with an over-activation of the NRLP3 inflammasome. These inflammatory diseases are for example the cryopyrin associated periodic syndromes (Agostini et al., 2004), rheumatoid arthritis (Van de Walle et al., 2014), obesity (Vandanmagsar et al., 2011) or Alzheimer's disease (Halle et al., 2008).

As used herein, a “subject” or an “individual” is an animal, preferably a mammal, including, but not limited to, human, dog, cat, cattle, goat, pig, swine, sheep and monkey. More preferably, the subject is a human subject. A human subject can be known as a patient.

As used herein, “subject in need” refers to an animal, preferably a mammal, more preferably a human, that suffer from, or is susceptible to suffer from any aetiology of ARDS, as explained above.

This subject may suffer three different stages of ARDS:
Early exudative stage (DAD)—The early exudative stage during the first 7 to 10 days is characterized by DAD. DAD is a nonspecific reaction to lung injury from a variety of causes. It is characterized by interstitial edema, acute and chronic inflammation, type II cell hyperplasia, and hyaline membrane formation.
Fibroproliferative stage—After approximately 7 to 10 days, a proliferative stage develops, characterized by resolution of pulmonary edema, proliferation of type II alveolar cells, squamous metaplasia, interstitial infiltration by myofibroblasts, and early deposition of collagen. It is unknown how long this phase lasts but is probably in the realm of two to three weeks.
Fibrotic stage—Some patients progress to a fibrotic stage, characterized by obliteration of normal lung architecture, fibrosis, and cyst formation. The degree of fibrosis ranges from minimal to severe.

Preferably, the subject in need to be treated by the modulators of the invention is in an early exudative or in a fibroproliferative stage of ARDS.

More preferably, said subject in need suffers from a virus-induced ARDS, more preferably from a Betacoronavirus-induced ARDS, even more preferably, from SARS-COV2-induced ARDS.

In this latter case, said subject is a “COVID suffering subject” i.e., an animal, preferably a mammal, more preferably a human, that suffers from COVID and/or has been diagnosed with COVID and/or is infected with at least one Betacoronavirus and/or suffers from at least one Betacoronavirus infection. Even more preferably, said subject is a “ COVID-19 suffering subject”, i.e., an animal, preferably a mammal, more preferably a human, that suffers from COVID-19 and/or has been diagnosed with COVID-19 and/or is infected with SARS-CoV2 and/or suffers from a SARS-CoV2 infection.

Diagnosis of a viral infection can be done by any known molecular means enabling to detect the presence of a virus in a biological sample (e.g., blood) of the subject. A number of diagnostic tools have been generated in the recent months to detect the SARS COV 2 virus specifically.

The tested subject is preferably suffering from a mild COVID form, displaying at least one of the symptom defined above (mild dry cough, mild muscle pain, mild headache, mild fever, mild fatigue, loss of taste or smell) and being diagnosed with COVID. He/she can also suffer from an asymptomatic form or from a strong COVID form, as defined above. In the first case, the treatment of the invention will permit to prevent the occurrence of the symptoms of the infection (mild dry cough, mild muscle pain, mild headache, mild fever, mild fatigue, loss of taste or smell). In the second case, the treatment of the invention will permit to alleviate the severe symptoms and diminish the risk of death for the patient.

The subjects to be treated can suffer only from said viral infection. They can also suffer from a comorbidity thereof, such as obesity, diabete, asthma, cancer or cardiovascular disease,

The compounds of the invention can be administered through various administration modes. The skilled person knowns how to select the most appropriate administration mode(s) depending on the compound, the disease and the subject to treat. For example, administration modes include, but are not limited to, as oral administration; administration by injection into a vein (intravenously, IV), into a muscle (intramuscularly, IM), into the space around the spinal cord (intrathecally), beneath the skin (subcutaneously, sc); sublingual administration; buccal administration; rectal administration; vaginal administration; ocular route; otic route; nasal administration; by inhalation; by nebulization in intensive mechanical circuit; cutaneous administration, either topical or systemic; transdermal administration.

In the context of the invention, the administration of the modulators of the invention is preferably performed by inhalation or by nebulization in an intensive mechanical circuit.

The present study reveals that the detection of the P2Y2-NLRP3 interaction positively correlated with disease severity and increased with viral infection. Accordingly, it is also possible to use the P2Y2-NLRP3 immune checkpoint as a prognostic marker of the future evolution of the COVID19 disease. As a matter of fact, they show that the detection of P2Y2-NLRP3 interaction in circulating blood cells is a prognostic marker for the transition between moderate to severe disease during COVID-19.

A further aspect of the invention therefore concerns a method to prognose the evolution of the COVID19 disease. Said method comprises the detection of the interaction between the P2Y2R and the NRLP3 proteins. When interaction of the two proteins is stimulated/enhanced in circulating blood cells in COVID19 patients, as compared with the level of interaction observed in blood cells obtained from control subjects, then a progression into a more severe disease is likely, as the viral infection tends to increase (because viral replication will be stimulated into host cells, and pro-inflammatory reprogramming of macrophages will take place).

Said control subjects are preferably subjects that are not infected by the SARS-COV-2 virus. They are more preferably healthy subjects.

The detection of the interaction between the P2Y2R and the NLRP3 proteins in circulating blood cells can be performed by any conventional means. It is for example possible to use proximity ligation assay as described in the experimental part below (see point 2.3.1.8.).

This detection step can be part of a screening test aiming at classifying the patients suffering from COVID19 disease, or aiming at evaluating what treatment would be the most appropriate for a patient (depending on the prognosed evolution of the COVID19 disease).

### Definitions in the Context of the Invention

As used herein, “viral infection” or “infection with a virus” or “virus infection” designates the fact that cells of an organism have been infected by at least one virus, the whole organism being said to suffer from a viral infection. In particular, “Viral infection due to SARS-CoV-2” or “SARS-CoV-2 infection” or “SARS-CoV2 infection” designates the fact that cells of an organism have been infected by the SARS-CoV-2 virus, the whole organism being said to suffer from said viral infection.

As used herein, the terms “prevent” or “preventing” or “prevention” or “prevention of the onset of a disease” means the reduction of the risk of appearing, of developing or of amplifying for a disease, for the causes of a disease, for the symptoms of a disease, for the effects (or consequences, preferably adverse, deleterious effects/consequences) of a disease, or any combination thereof; and/or delaying the onset, development or amplification of a disease, the causes of a disease, the symptoms of a disease, the effects (or consequences, preferably adverse, deleterious effects/consequences) of a disease, or any combination thereof. In the present case, “preventing COVID disease” comprises reducing the likelihood that the patient undergoes the switch into a severe form.

As used herein, the terms “treat”, “treating”, “treatment” and the like mean the reduction, inhibition, amelioration, stabilization and/or disappearance of a disease (or an ailment, or a condition), of the causes of a disease, of the symptoms (or signs) of a disease, of the effects (or consequences, preferably adverse, deleterious effects/consequences) of a disease (e.g. COVID such as COVID-19, and/or symptoms associated therewith), fighting the disease, or any combination thereof. It will be appreciated that, although not precluded, treating a disorder or condition does not require that the disorder, condition or symptoms associated therewith be completely eliminated. For examples, treating results in the reduction of at least one sign or symptom of the disease or condition. Treatment includes (but is not limited to) administration of a therapy, and may be performed either prophylactically, or subsequent or the initiation of a pathologic event. Treatment can require administration of a therapy more than once.

More specifically, “treating a Betacoronavirus infection” or “treating COVID” refers to fighting at least one Betacoronavirus infection in a human or animal organism. Advantageously, the rate of viral infection (infectious titre) in the organism will decrease, and the Betacoronavirus will completely disappear from the organism within a shorter period of time than expected without treatment. The terms “treating a Betacoronavirus infection” or “treating COVID” also refer to reducing/inhibiting/ameliorating/stabilizing/making disappear, the symptoms/signs associated with a Betacoronavirus infection (respiratory syndrome, kidney failure, fever, etc.). More specifically, “treating BARS-CoV-2” or “treating COVID-19” refers to fighting the SARS-CoV-2 infection in a human or animal organism. Advantageously, the rate of viral infection (infectious titre) in the organism will decrease, and the SARS-CoV-2 will completely disappear from the organism within a shorter period of time than expected without treatment. The terms “treating SARS-CoV-2” or “treating COVID-19” also refer to reducing/inhibiting/ameliorating/stabilizing/making disappear, the symptoms/signs associated with the SARS-CoV-2 infection (respiratory syndrome, kidney failure, fever, etc.).

By a “therapeutically effective amount” is meant a sufficient amount of the compound of the invention (e.g. P2Y2 receptor agonist or the inhibitor of P2X receptor activity) at a reasonable benefit/risk ratio applicable to the medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1,0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The term “pharmaceutically” or “pharmaceutically acceptable” refers to molecular entities and compositions that do not produce an adverse, allergic or other untoward reaction when administered to a mammal, especially a human, as appropriate. A “pharmaceutically acceptable carrier” or “excipient” refers to a non-toxic solid, semi-solid or liquid filler, diluent, encapsulating material or formulation auxiliary of any type. In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermat, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellutose. Dispersions can also be prepared in glycerol, liquid polyethylene glycols, and mixtures thereof and in oils. Under ordinary conditions of storage and use, these preparations contain a preservative to prevent the growth of microorganisms. The active ingredient can be formulated into a composition in a neutral or salt form. Pharmaceutically acceptable salts include the acid addition salts (formed with the free amino groups of the protein) and which are formed with inorganic: acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride, Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The terms “pharmaceutically acceptable salt”, as used herein, mean a salt of a compound which is pharmaceutically acceptable, as defined above, and which possesses the pharmacological activity of the corresponding compound. The pharmaceutically acceptable salts comprise:
(i) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(ii) base addition salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

In its broadest sense, a “vector” as used herein is any vehicle capable of facilitating the transfer of the antisense oligonucleotide siRNA or ribozyme nucleic acid to the cells and preferably cells expressing the targeted proteins (e.g. P2X receptor). Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell tined with plasmid, production of recombinant retroviruses by the packaging cell line, collection of viral particles from tissue culture media, and infection of the target cells with viral particles) are provided in Kriegter, (1990) and in Murry and Wolk, (1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUCl9, pRC/CMV, S V40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

The invention will be further illustrated by the following figures and examples, However, these examples and figures should not be interpreted in any way as limiting the scope of the present invention.

### DESCRIPTION OF THE FIGURES

FIG. 1 shows that P2Y2 agonists inhibit IL-1β secretion detected in response to LPS+ATP stimulation and IFNγ-mediated macrophage pro-inflammatory reprogramming. (A, B) PMA-THP1 macrophages were incubated with 50 μM of MR52768 (during 1 hour) (A) or indicated concentrations of Diquafosol (during 12 hours) (B). Cells were then stimulated with 10 ng/ml LPS (during 3 hours) and 5mM ATP (during 6 hours). IL-1β release was detected in the supernatant of treated cells by western blot. (C, D) PMA-THP1 macrophages were incubated with indicated concentrations of UTP (C) or Diquafosol (D) and stimulated with 50 ng (C) or 20 ng (D) of IFNγ during 24 (C) or 12 (D) hours. RFS expression was analyzed by western blot. Representative experiments of 3 independent experiments are shown.

FIG. 2 demonstrates that purinergic receptors dictate viral replication. Vero E6 cells were infected with SARS-CoV-2 during 2 hours (A, D), 16 hours (B, E) and 24 hours (Figures C, F) with 500 μM UTP, 100 μM Diquafosol, 5 μM Denufosol, 50 μM PPADS or 100 μM BzATP and mRNA expression of RdRp (A-C) and E (D-F) were analyzed using quantitative RT-PCR.

FIG. 3 discloses how purinergic: receptors control cytopathogenicity elicited by SARS-CoV-2. Vero E6 cells were infected with SARS-CoV-2 during 72 hours in presence of indicated concentrations of UTP (A), Diquafosol (B), Denufosol (C) or BzATP (D) and cytopathogenicity was analyzed using MTT assay. Absorbances at 570 nm of representative experiments are shown.

FIG. 4 shows that P2Y2 and NLRP3 interaction is enhanced during SARS-CoV-2 infection and COVID-19.
(A to F) Representative images of P2Y2-NLRP3 PLA+cells and frequencies detected on neutrophils (A, B), CD14+ monocytes (C, D) and CD3+ T cells (E, F) obtained from SARS-CoV-2-infected and uninfected patients are shown. Images are representative of 18 SARS-CoV-2-infected and 7 uninfected patients for neutrophils (A), and 14 SARS-CoV-2-infected and 6 uninfected patients for CD14+ monocytes (C) and CD3+ T cells (E). Patients with moderate (green) and severe (red) disease are shown. Scale bars, 5 μm (A), 3 μm (C) and 2 μm (E). (G, H) Representative images of P2Y2-NLRP3 PLA+ cells (G) and frequencies (H) detected on BALFs obtained from SARS-CoV-2-infected and uninfected NHP are shown. Images are representative of 5 SARS-CoV-2-infected and 4 uninfected NHP. Scale bar, 5 μm. (I, J) Representative images of P2Y2-NLRP3 PLA+ cells (I) and frequencies detected on SARS-CoV-2-infected and uninfected ACE2-A549 cells are shown. Scale bar, 10 μm. Images are representative of three independent experiments. Data are presented as means±SEM. Unpaired two-tailed Mann-Whitney (B, D, F, H) or unpaired two-tailed t (J) tests were used. *P<0.05; **P<0.01 and ****P<0.0001.

FIG. 5 demonstrates the increased plasma IL-1 β secretion and pyroptosis of alveolar macrophages in P2y2^{-/-} mice.
(A to G) Plasma IL-1β(A), representative flow cytometry analysis (B), frequencies of bronchoalveolar CD11b⁺GR1⁻F4/80⁺CD11c^{high}CD40^{high} macrophages (C), representative images (D, F) and frequencies (E, G) of cleaved Caspase-1⁺ (CASP1⁺) CD40⁺ macrophages (D, E)) or TUNEL⁺CD40⁺ macrophages (F, G) were determined from 7 to 8 wild-type P2y2^{+/+} mice and 8 to 9 P2y2^{-/-} mice. (H) Kaempferol (Kaempf.)-treated, control, NLRP3- or CASP1-depleted PMA-THP1 macrophages were analyzed for LDH release after stimulation with LPS and ATP. Nuclei and cellular shapes are detected using Hoechst 33342 and phase contrast in (D) and (F). Scale bars in (D) and (F) are 5 μm. Data are presented as means ±SEM. Unpaired two-tailed Mann-Withney test (A, C, E and G) and two-way ANOVA analyses (H) were used. *P<0.05, **P<0.01 and ****P<0.0001.

FIG. 6 shows how P2Y2 negatively regulates pro-inflammatory functions of macrophages.
(A, B) Representative images of P2Y2-NLRP3 PLA+ cells detected in PMA-THP1 macrophages (A) and MDMs cells (B) treated with IFNγ or LPS (A) during 72 hours are shown. (C, D) Frequencies of PLA+ cells detected on treated PMA-THP1 macrophages (C) and MDMs cells (D) are shown. (E to P) Control or LPS+ATP-stimulated PMA-THP1 macrophages (E, F, I, J, P) or MDMs (G, H, K and L-O) treated with Suramin (E), OxATP (E), Kaempferol (Kaempf.) (E, G and J-N), Diquafosol (Diqua.) (I, P), IFNγ (3, N and P) or expressing shRNA (F) or transfected with siRNA (H and O) were analyzed for IL-1β and IL-10 (E-K), membrane CD163 (L and M) and IRF5 expressions (N-P), and gene expression of known human polarization-specific markers (Q). Results were obtained from at least 3 independent experiments. Representative images and western blots are shown. Data are presented as means±SEM. Unpaired one-tailed Mann-Withney test (D and K), paired Wilcoxon two-tailed test (M), unpaired two-tailed t-test (J), one-way (C) and two-way (E and F) ANOVA analyses were used. *P<0.05; **P<0.01, ***P<0.001 and ****P<0.0001.

FIG. 7 discloses that the purinergic receptors P2Y2 and P2X7 dictate susceptibility to SARS-CoV-2 infection through the modulation of viral replication.
(A-C) Vero E6 cells (A, C) and ACE2-A549 cells (B) were infected with SARS-CoV-2 (MOI=0.1 or 0.2) during indicated hours post-infection (hpi) and evaluated for P2Y2 (A, B), P2X7 (C), NLRP3 (A, B), Spike (A-C), GAPDH (A, C) and □-Tubulin (B) expressions. (D-K) Vero E6 cells (D, E, I-K) and ACE2-A549 cells (F-H) were respectively infected with SARS-CoV-2 (MOI=0.1 or 0.2) during 24 (D, E, 1-K) or 48 (F-G) hours in presence of 500 μM UTP, 100 μM Diquafosol (Diqua.), 5 μM Denufosol (Denu.), 50 μM PPADS, 100 μM OxATP or 100 μM BzATP. Intracellular mRNA expressions of RdRp (D, F) and E (E, G) were analyzed using quantitative RT-PCR (D-G); Spike K), ACE2 (K) and GAPDH (H, K) expressions were determined by western blot; Spike positive cells were imaged (I) and quantified (J) using fluorescence microscopy. Representative western-blots (A-C, H and K) and images (I) of 3 independent experiments are shown. Fold changes of RdRp and E RNA from SARS-CoV-2 infected Vero E6 cells (n=3) and ACE2-A549 cells (n=2-3) are shown (D-G). Scale bar in (I) is 10 μm. Data are presented as means±SEM. One-way (D-G) and two-way (J) ANOVA analyses were used. **P<0.01, ***P 0.001 and ****P<0.0001.

FIG. 8 shows the pathological changes and NLRP3 expression in the lungs of COVID-19 patients with severe disease.
(A) Parenchymal multifocal damage (with inflammation and fibrous proliferative phase) and hyperplasic amphophilic type II pneumocytes are detected in COVID-19 patients as compared to non COVID-19 patients (staining with hematoxylin and eosin; bars, 22 μm and 50 μm). (B) NLRP3 expression in mononuclear cells is detected into alveolar septa and lumen in both COVID-19 and non-COVID-19 patients (bars, 45 μm and 50 μm). (C) Type II pneumocytes with cytoplasmic NLRP3 expression (bar, 3 μm). (D) NLRP3 positive alveolar macrophages in lumen (bar, 3 μm). (E-G) Syncytium detected on COVID-19 patients express macrophage marker CD68 (E) (bars, 8 μm and 3 μm), NLRP3 (F) (bars, 8 μm and 3 μm) and cytoplasmic double strand RNA, indicative of viral infection (G) (bars, 12 μm and 4 μm). Magnifications are shown.

FIG. 9 discloses the validation of the Caspase-1 knockdown. Caspase-1 (CASP1) expression of control (shCo.) and CASP1 (shCASP1-1 and -2)-depleted, PMA-THP1 macrophages were determined by western blot. Representative western blots of 3 independent experiments are shown.

FIG. 10 shows the effect of AR-C118925XX on the P2Y2-NLRP3 interaction and validation of P2Y2 knockdowns. (A) Frequency of P2Y2-NLRP3 PLA+ cells detected after treatment of PMA-primed THP1 with 100 μM AR-C118925XX during 96 hours is shown. (B, C) P2Y2 expression of control (shCo. or siCo.), stably P2Y2 (shP2Y2)-depleted PMA-primed THP1 macrophages (B) or siP2Y2-depleted MDMs (C) were determined by western blot after tentiviral transduction (B) and after transient transfection with SMART POOL P2Y2 siRNA (C). Representative western blots of 3 independent experiments are shown. Data are presented as means±SEM. Unpaired two-sail t-test was used. ****P<0.0001.

FIG. 11 demonstrates that the NLRP3 protein represses LPS+ATP-elicited macrophage migration.
(A) PMA-primed P2Y2- or NLRP3-depleted-THP1 cells were analyzed for migration after LPS+ATP stimulation. Results shown are fold changes with respect to the control cells and were obtained from 5 independent experiments. Data are presented as means±SEM. (B-D) PMA-primed THP1 control or depleted for NLRP3 were stimulated with LPS+ATP and analyzed for F-actin polymerization (with Phalloidin) and PYK2Y402* using confocal microscopy. Scale bars of representative images shown for shControl and shNLRP3 THP1 cells are 2 μm and 20 μm, respectively. Scale bar of magnification is 13 μm. Representative images of three independent experiments are shown (B). Mean fluorescence intensities of Phalloidin (C) and PYK2Y102* (D) are shown. Results shown were obtained from 3 independent experiments. Data are presented as means±SEM in (A), (C) and (D) and analyzed with one-way (A) and two-way (C and D) ANOVA tests, Significances are **P<0,01, ***P<0.001 and ****P<0,0001.

FIG. 12 shows the effects of P2Y2 agonists on the SARS-CoV-2-mediated cytopathogenic effects and viability of Vero E6 cells and ACE2-A549 cells.
(A-H) Vero E6 cells (A-E) and ACE2-A549 cells (F-H) were infected or not with SARS-CoV-2 (MOI=1 or 2) during 72 hours in presence of indicated concentrations of Diquafosol (Diqua.) (A, D, G), Denufosol (Denu.) (B, E, H) or UTP (C, F). Cytopathogenicity and/or cell survival were then analyzed using MIT assays. Results were obtained from 3 independent experiments. Data are presented as means±SEM. One-way ANOVA analyses were used. Significances are *P<0.05 and **P<0.01.

FIG. 13 shows the effects of purinergic receptor modulators agonists on the SARS-CoV-2-mediated cytopathogenic effects and viability of Vero E6 cells and ACE2-A549 cells.
(A) Vero E6 cells were infected with SARS-CoV-2 (MOI=0.1 or 0.2) during 24 hours in presence of 100 μM OxATP and Spike expression was determined using fluorescence microscopy. Representative image of 3 independent experiments are shown. Scale bar is 10 μm. (B-I) Vero E6 cells (B-D, G, H) and ACE2-A549 cells (E, F, I) were incubated during 72 hours in presence of indicated concentrations of PPADS (B, C, E), OxATP (D, F), and BzATP (G-I). Cytopathogenicity and/or cell survival were analyzed using MTT assays. Results were obtained from 3 independent experiments. Data are presented as means±SEM. One-way ANOVA analyses were used. Significances are *P<0.05, **P<0.01 and ***P<0.001.

FIG. 14 displays the effects of purinergic receptor modulators on ACE2 membrane expression. (A, B) Vero E6 cells (A) and ACE2-4549 cells (B) were treated during 24 hours with 500 μM UTP, 100 μM Diquafosol (Diqua.), 5 μM Denufosol (Denu.), 50 μM PPADS, 100 μM OxATP or 100 μM BzATP. Frequency of ACE2+cells was then analyzed using Guava easyCyte 6HT2L flow cytometer (Luminex). Results shown were obtained from 3 independent experiments. Data are presented as means±SEM. One-way ANOVA analyses were used.

FIG. 15 displays the effect of a modulator of ATP (apyrase) and probenecid on SARS-COV-2 infection. ACE2-A549 cells were infected during 48 hours with SARS-CoV-2 in presence or in absence of 5 UI/mL Apyrase (A, C) or 500 μM Probenecid (B, D). Cells were analyzed for Spike S expression by fluorescent microscopy (A, B) and for cell viability (C, D). Percentages of Spike+ cells and cell survival are shown (n=3). Data are presented as means±SEM. Student-t test was used. *P≤0.05 and ***P≤0.001 and ****P<0.0001.

FIG. 16 displays the effect of the NLRP3 inhibitor Tranilast on SARS-COV-2 replication. Caco-2 (A, B, D) and ACE2-A549 (C, E) cells were infected during 48 hours with SARS-CoV-2 in presence or in absence of 100 μM Tranilast. Spike expression (A), fold change in E RNA expression (B, C) and cell survival without infection (D, E) were analyzed as described in Materials and Methods, and shown (n=3). Data are presented as means±SEM. Student-t test was used. *P≤0.05.

FIG. 17 displays the effect of the P2Y2 agonist Diquafosol on Bleomycin-induced lung inflammation. (A) Pulmonary Lesions detected on treated mice (day 20) were analyzed using CT scan. Representative images of CT are shown and reveal that Diquafosol treatment reduces lung lesions induced by bleomycin treatment. (B) Intersitial CD45+CD11b+Ly6G−Ly6C−CD64+ macrophages were detected using flow cytometry and shown. Data are presented as means±SEM. One way anova test was used. *P≤0.05 and **P≤0.001.

### EXAMPLES

### 1. Materials and Methods

1.1. Cell lines and SARS-COV-2

Monocytic THP1 cells were obtained from ATCC and were maintained in RMPI-1640-Glutamax medium supplemented with 10% heat inactivated fetal bovine serum (FBS) and 100 UI/mL penicillin-streptomycin (Life technology). THP1 macrophages were obtained by treatment for 3 hours with 100 nM phorbol-12-myristate-13-acetate (PMA, Invivogen) of THP1 monocytes and after extensive washings were let to differentiate for 72 hours before experimentation. The African green monkey kidney epithelial (Vero E6) cells were purchased from ATCC (ATCC CRL-1587) and cultured in Dulbecco's modified Eagle's medium (DMEM, Gibco, USA) with 10% fetal bovine serum (FBS), 100 U/mL penicillin, and 100 μg/mL streptomycin at 37° C. All cell lines used were mycoplasma-free. SARS-CoV-2 was propagated on Vero E6 cells in a biosafety level-3 (BLS-3) laboratory. After 72 hours of infection with a multiplicity of infection of 0.2, the supernatant was collected and centrifuged during 5 minutes at 1500 rpm at 4° C. to remove cellular debris. Then, supernatant was centrifuged during 20 minutes at 3000 rpm at 4° C. and stored at −20° C. Viral titration was performed by determining cytopathogenic effects associated with viral infection. Cell lysis was determined using agarose-containing semi-solid medium (Björn Meyer; Institut Pasteur). LPS, ATP, UTP, Diquafosol and Denufosol, pyridoxal phosphate-6-azophenyl-2′,4′-disulfonic acid (PPDAS) and P2X7 receptor agonist 2′,3′-O-(4-benzoyl-benzoyl)ATP (BzATP) were obtained from Sigma, Diquafosol from Clinisciences and Denufosol from Carbosynth.

### 1.2. Western Blots

Human THP-1 cells were cultured in RPMI 1640 media, supplemented with 10% FBS and differentiated by treatment for 3 hours with 100 nM phorbol-12-myristate-13-acetate (PMA, Invivogen). After 2 days, macrophage THP-1 cells were stimulated first 3 hours with ultrapure LPS from E. coli (10 ng/ml, LPS) and then stimulated for 6 hours with ATP (5 mM, Sigma) or treated with 50 ng or 20 ng of IFNg during 24 or 12 hours as indicated. Then, supernatants and cells were collected for western blot analysis.

Cells or supernatant were lysed in appropriated buffer (250 mM NaCl, 0.1% NP-40, 5 mM EDTA, 10 mM Na3VO4, 10 mM NaF, 5 mM DTT, 3 mM Na4P2O7, 1 mM EGTA, 10 mM Glycerol phosphate, 10 mM Tris-Hcl (pH=7.5) and the protease and phosphatase inhibitors (Roche)). Equal amount of supernatant or 10-40 μg of protein extracts were run on 4-12% or 10% SDS-PAGE and transferred at 4° C. onto a nitrocellulose membrane (0.2 Micron). After incubation for 2 hours at room temperature with 5% nonfat milk or BSA (Bovine Serum Albumine) in Tris-buffered saline and 0.1% Tween 20 (TBS-Tween), membranes were incubated with primary antibody at 4° C. overnight. Horseradish peroxidase-conjugated goat anti-mouse or anti-rabbit (SouthernBiotech) antibodies were then incubated for 1 hour 30 minutes and revealed with the enhanced ECL detection system (GE Healthcare). The primary antibodies against. IL-1β, IRF5 and GAPDH were from Abcam and Horseradish peroxidase-conjugated goat anti-rabbit (SouthernBiotech) antibodies were incubated for 1 hours and revealed with the enhanced ECL detection system (GE Healthcare). Western blots shown are representative of at least of three independent experiments.

1.3. Cytopathogenic assay

The cytotoxic tests were performed using Vero E6 cells. Twenty-four hours before infection, 4×10³ cells were seeded per well on 96 well plates. Cells were pretreated with indicated concentrations of UTP, Dequifosol, Denufosol and BzATP during 4 hours before infection and infected with a multiplicity of infection between 1 and 2. Viability of cells was then determined after 72 hours of infection using (bromure de 3-(4,5-dimethylthiazol-2-yl)-2,5-diphenyl tetrazolium) (MTT) assay following manufacter's instructions.

### 1.4. Quantitative RT-PCR

After infection, total cell RNA was extracted with the RNeasy minikit (QIAGEN) according to the manufacturer's instructions. RNA was recovered according to the manufacturer's instructions for total cell RNA extraction. Quantifications were performed by real-time PCR on a Light Cycler instrument (Roche Diagnostics, Meylan, France) using the second-derivative-maximum method provided by the Light Cycler quantification software, version 3.5 (Roche Diagnostics). Standard curves for Sars-Cov2 RNA quantifications were provided in the quantification kit and were generated by amplification of serial dilutions of the provided positive control. Sequences of the oligonucleotides and probes used to quantify the RdRp and E genes are given below.

For the RdRp gene the LightMix®Modular Wuhan CoV RdRP-gene530 (Cat.-No. 53-0777-96, Tib Mol biol) was used with the following primers and probe:
RdRp SARSr-F 5′: gTgARATggTCATgTgTggCgg (SEQ. ID NO:3);
RdRp SAR5r-R 5′: CARATgTTAAASACACTATTAgCATA (SEQ ID NO :4);
RdRp SARSr-P2 5′: 6FAM-CAggTggAACCTCATCAggAgATgC-BBQ (6FAM-SEQ ID NO:5-BBQ).

For the E-gene:
E Sarbeco P1 5′: 6FAM-ACACTAgCCATCCTTACTgCgCTTCg-BBQ (6FAM-SEQ ID NO:6-BBQ):
E Sarbeco F 5′:ACAggTACgTTAATAgTTAATAgCgT (SEQ ID NO:7);
E Sarbeco R 5′: ATATTgCAgCAgTACgCACACA (SEQ ID NO:8).

Conditions used for the amplification of RdRp and E genes are the same and are as follow: Reverse transcription (5 minutes), denaturation (5 minutes) and 45 cyles with the following steps (95° C., 5 s−60° C., 15 s−72° C. 15 s). Results were normalized by the total amount of RNA in the sample and also reported to the condition without any compound (wild-type condition).

1.4. In Vitro Infection with SARS-CoV-2

ACE2-A549 and Caco2 cells were infected for 48 hours with SARS-CoV-2 (BetaCoV/France/IDF0372/2020) at multiplicity of infection (MOI) ranging from 0.1 to 6 in absence or in presence of 5 UI/mL Apyrase (Sigma), 500 μM of c(Sigma) or 100 μM of Tranilast (Sigma).

### 1.5. Immunofluorescence

After viral infection with a MOI of 6, cells were fixed at room temperature during 20 min with 4% paraformaldehyde (PFA), permeabilized with 0.3% Triton-X100 and blocked with 10% FBS for 1 hour at room temperature, Then, cells were stained for 2 hours with mouse anti-Spike S antibody (#GTX632604, Genetex) and 1 hour with goat anti-mouse IgG conjuguated to Alexa Fluor 546 (#A11030, Invitrogen). Nuclei were also stained with Hoechst 33342 (#H3570, Invitrogen) as previously described. Cells were analyzed by fluorescent microscopy on Leica DMI8.

### 1.6. Flow Cytometry

Mice were sacrificed and the right lungs were removed, washed in cold PBS, minced, and digested using the lung dissociation kit (Mittenyi, #130-095-927) for 30 min under agitation at 37° C. After enzyme digestion, lung tissue was passed through a 70 μm filter and red blood cells were lysed with ACK lysing buffer (#A10492-01, Gibco) for 10 min on ice. Then, cells were washed once in DMEM medium than in PBS. Cells were incubated with purified anti-mouse CD16/32 (#101302, BioLegend) for 10 minutes at 4° C. For membrane staining, anti-CD45APC-Vio770 (#130-110-662, miltenyi Biotec), anti-Ly6G PerCP-Vio 700 (#130-117-500, Miltenyi Biotec), anti-CD169 PE (#130-104-953, Miltenyi Biotec), anti-CD11c PE-Vio 770 (#130-110-703, Miltenyi Biotec), anti-CD11b BUV395 (#563553, B D Horizon), anti-Ly6C AF700 (#128024, BioLegend), anti-CD64BV605 (139323, BioLegend) and anti-Siglec-F PE-CF594 (#562757, B D Horizon) antibodies were incubated for 20 min at 4° C. Cells were fixed with 4% PFA. Samples were acquired on an LSR Fortessa II (B D, Franklin Lakes, NJ) with FACSDiva software, and data were analyzed with FlowJo 10.0.7 software (Tree Star, Inc., Ashland, OR).

### 1.7. Celt Viability Assay

Cell viability was determined after 48 hours using 3-(4,5-dimethylthiazol-2-yl)-2,5-diphenyltetrazolium bromide (MTT) assay (#M5655, Sigma) following manufacturer's instructions.

### 1.8. Western Blotting

After infection with a MOI of 2, total cellular proteins were extracted lysed in appropriated buffer containing 1% NP40, 20 mM HEPES, 10 mM KCl, 0.5 M EDTA, 10% Glycerol, protease and phosphatase inhibitors for 30 min at 4′C. After centrifugation, supernatants were collected, subjected to protein quantification and 15 to 20 μg of protein were loaded. A classical western blotting was then performed.

### 1.9. Quantitative RT-PCR for SARS-COV-2

After infection with a MOI of 0.1, total cell RNA was extracted using QIAshredder kit (#79654, Qiagen) and the RNeasy kit plus Mini kit (#74136, Qiagen) according to the manufacturer's instructions. RNA was recovered according to the manufacturer's instructions for total cell RNA extraction. Quantifications of SARS-COV-2 E RNA were performed by real-time PCR on a Light Cycler instrument (Roche Diagnostics, Meylan, France) using the second-derivative-maximum method provided by the Light Cycler quantification software (version 3.5 (Roche Diagnostics)). Standard curves for SARS-CoV-2 RNA quantifications were provided in the quantification kit and were generated by amplification of serial dilutions of the provided positive control.

### 1.10. Mouse Model of Bleomycin-Induced Lung Inflammation

Seven weeks old C57BL/6 mice were obtained from Janvier laboratories, maintained on 12 h dark/light cycles and provided with water and standard rodent diet ad libitum. Pulmonary inflammation was induced in C57BL/6 mice by intratracheal instillation of 50 UI of bleomycin. Every 3 days, 300 μg of Diquafosol were administrated intraperitoneally in mice. Mice were sacrificed at 21 days after bleomycin instillation.

### 1.11. Computerized Tomography (CT) Scan

Lung CT scan was acquired 1 day before the sacrifice using Bioimaging IVIS Spectrum-CT (PerkinElmer) and the Living Image Software 4.3 (PerkinElmer).

### 1.12. Quantification and Statistical Analysis

All values were expressed as the mean±SEM of cell individual samples.

Student t-test and one-way ANOVA test were used for statistical analysis. Statistical data were analyzed with Graphpad prism 8 software. Statistical significance was given as *P≤0.05, **P≤0.001 and ****P<0.0001.

### 2. Results

### 2.1. P2Y2 Agonists Inhibit NLRP3-Dependent IL-1β Secretion and Macrophage Pro-Inflammatory Reprogramming

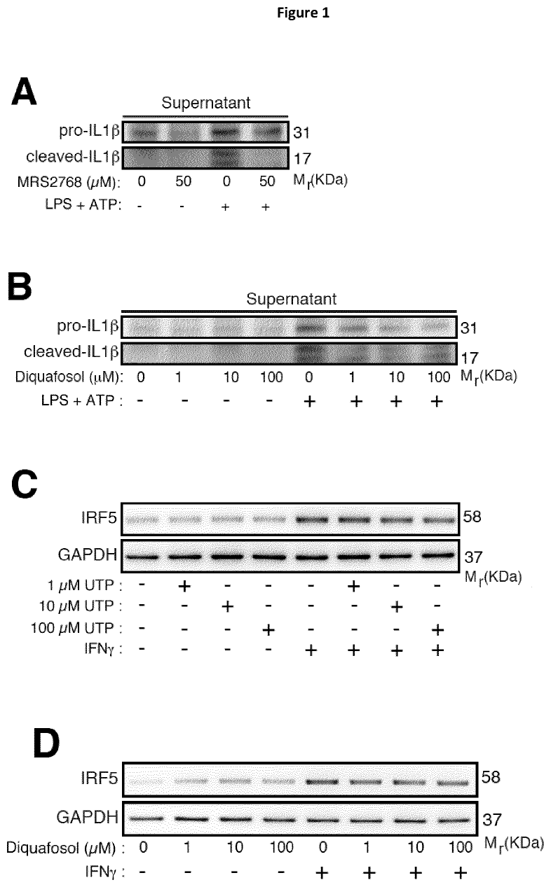

To study the effects of P2Y2 agonists on NLRP3 inflammasome activation, PMA-treated THP1 macrophages were analyzed for IL-1β secretion after pretreatment with 50 μM of P2Y2 agonist MRS2768 during 1 hour and stimulation with 10 ng/ml LPS during 3 hours and 5 mM ATP during 6 hours (LPS+ATP). As previously described in many studies, stimulation of PMA-treated THP1 macrophages with LPS+ATP led to a robust secretion of IL-1 β in the supernatant of treated cells (FIG. 1A). However, treatment of cells with MRS2768 strongly inhibited the release of IL-1β (FIG. 1A), thus demonstrating that the activation of P2Y2 with MR52768 represses NLRP3 inflammasome activation.

Then, PMA-treated THP1 macrophages were simulated with LPS+ATP in presence of indicated concentrations of Diquafosol, which is a P2Y2 agonist used for the treatment of cystic fibrosis lung disease (Kellerman et al., 2002) and supernatants were analyzed for IL-1β release. As shown with MRS2768 (FIG. 1A), Diquafosol also impaired the release of IL-1β in the supernatant of treated macrophages (FIG. 1B).

Considering that NLRP3 was also shown to contribute to macrophage pro-inflammatory reprograming (Camell et al., 2015), PMA-treated THP1 macrophages were also stimulated with IFNγ during 24 hours in presence or in absence of different concentrations of the natural P2Y2 agonist uridine-5′-triphosphate (UTP) and the expression of Interferon regulatory factor 5 (IRF5), which is a central transcription factors of macrophage pro-inflammatory reprogramming (Krausgruher et al., 2011³⁵) was determined. A decrease of IRFS expression was detected in presence of 10 and 100 μM of UTP (FIG. 1C). These results were confirmed with indicated concentrations of Diquafosol (FIG. 1D), thus suggesting that P2Y2 agonists also inhibit pro-inflammatory reprogramming of macrophages. Collectively, these data demonstrate that P2Y2 agonists repress NLRP3-inflammasome activation and macrophage pro-inflammatory reprogramming.

### 2.2. Purinergic Receptors Control Viral Replication of SARS-CoV-2 and Associated Cytopathogenic Effects

Considering that purinergic receptor P2Y2 and other purinergic receptors (such as purinergic receptor P2X7) may regulate permissivity to viral infection (Séror et al., 2011²²; Paoletti et al., 2019²⁰), the impact of P2Y2 agonists (UTP, Diquafosol and Denufosol), of P2X receptor antagonist pyridoxal phosphate-6-azophenyl-2′,4′-disulfonic acid (PPDAS) and of P2X7 receptor agonist 2′,3′-O-(4-benzoyl-benzoyl)ATP (BzATP) was next determined on the replication of SARS-CoV-2.

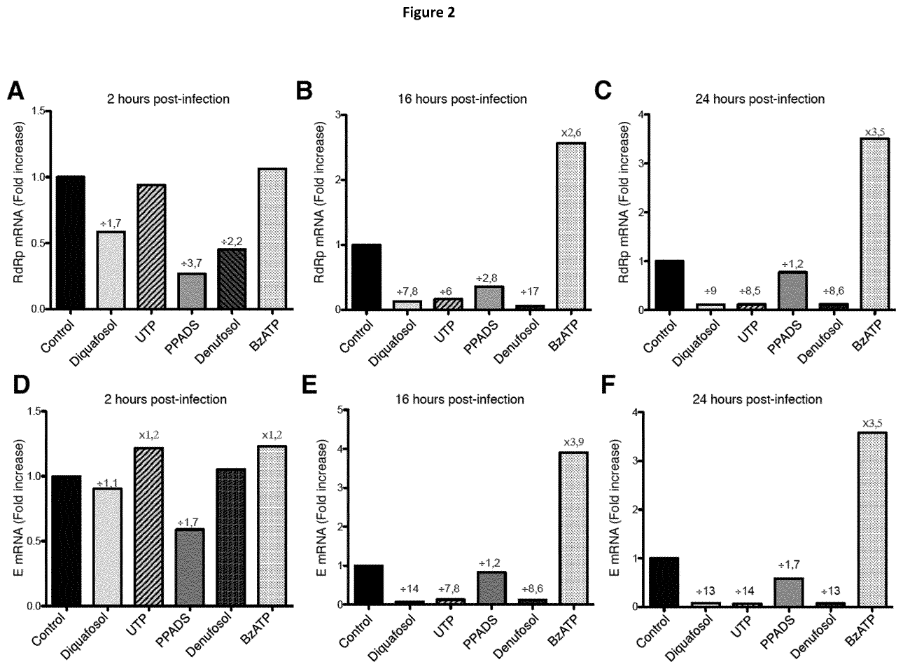

African green monkey kidney epithelial (Vero E6) cells were infected with SARS-CoV-2 during 2 hours (FIGS. 2A and 2D), 16 hours (FIGS. 2B and 2E) and 24 hours (FIGS. 2C and 2F) in presence of 500 μM UTP, 100 μM Diquafosol, 5 μM Denufosol, 50 μM PPADS and 100 μM BzATP.

To study viral replication, mRNA expression of RNA-dependent RNA polymerase (RdRp) and E genes were analyzed using quantitative RT-PCR. The activation of the P2Y2 by UTP, Diquafosol and Denufosol strongly reduced the amount of RdRp and E mRNA in Vero cells after 16 hours (FIGS. 2B and 2E) and 24 hours (FIGS. 2C and 2F) of infection (FIGS. 2A-2F), as compared to control, indicating that the purinergic receptor P2Y2 acts as a restriction factor for SARS-CoV-2 infection.

Interestingly, PPDAS also repressed RdRp and E mRNA expression after 16 (FIGS. 2B and 2E) and 24 (FIGS. 2C and 2F) hours of infection and P2X7 receptor agonist 2′,3′-O-(4-benzoyt-benzoyl)ATP (BzATP) strongly enhanced viral replication at these time points. These results revealed that purinergic receptors P2X and in particular, P2X7 favor the replication of SARS-CoV-2.

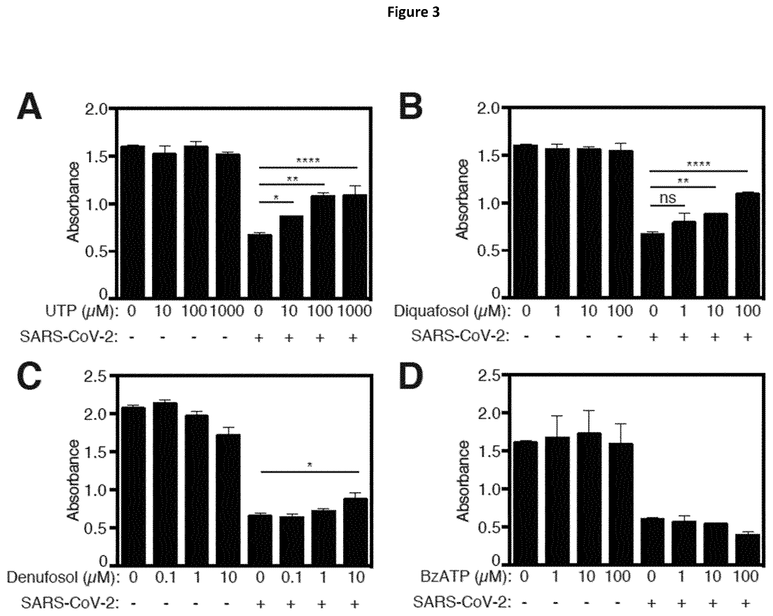

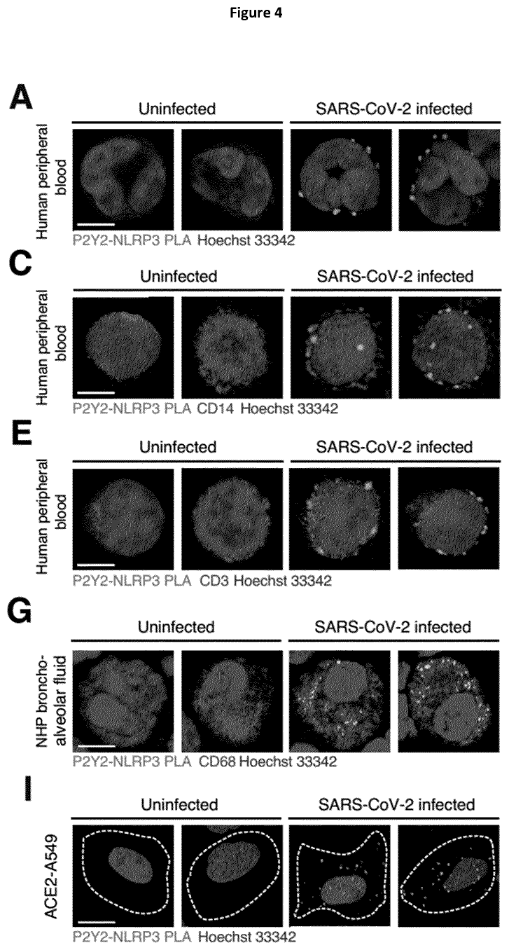

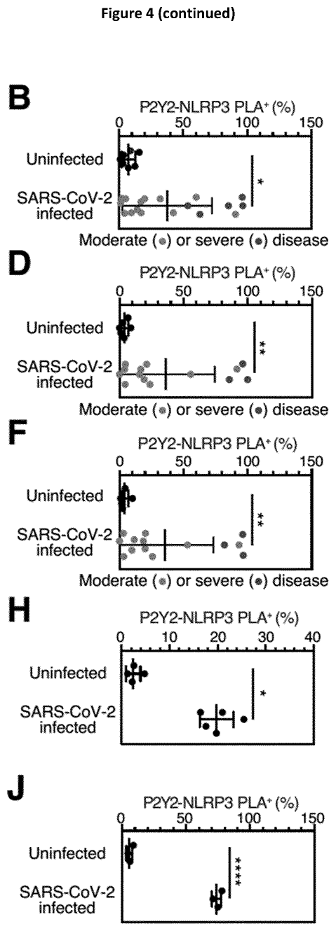

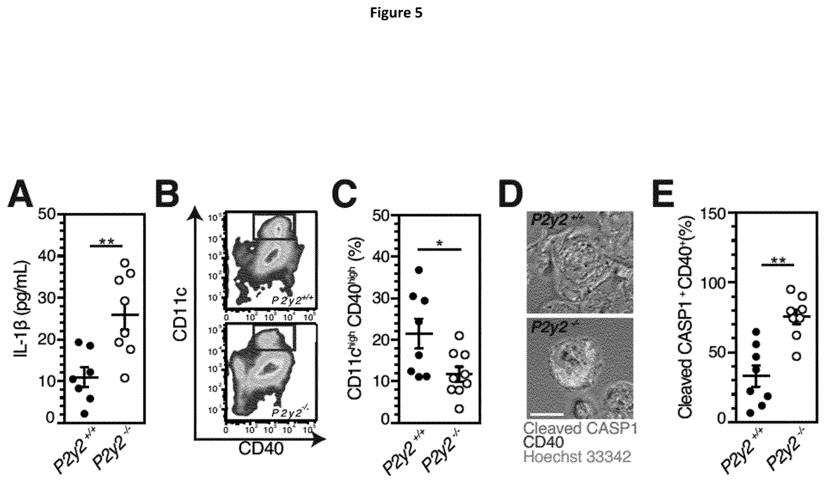

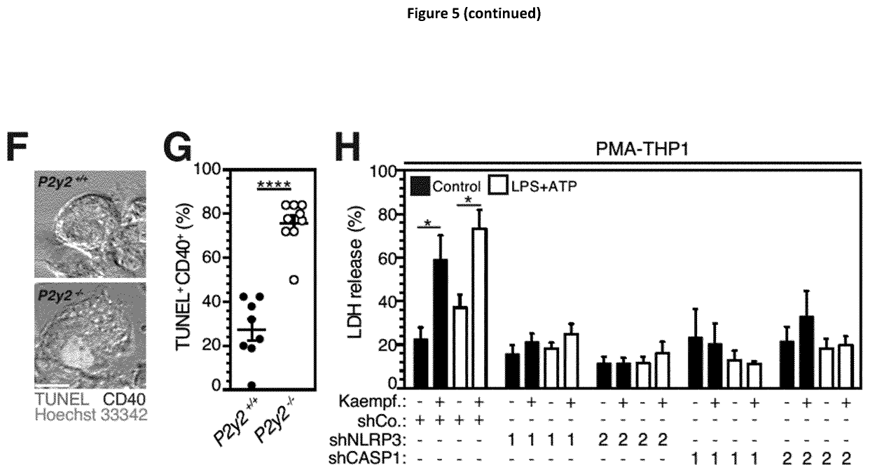

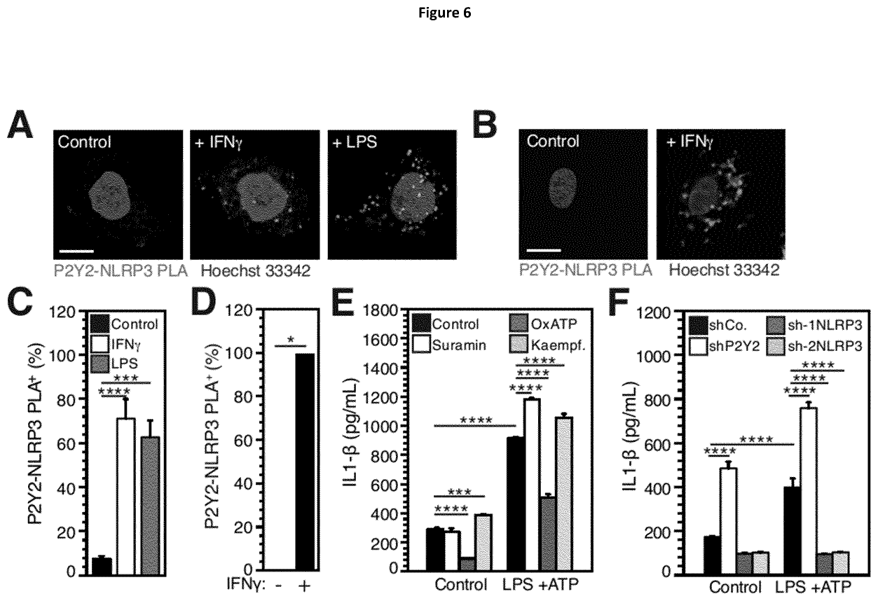

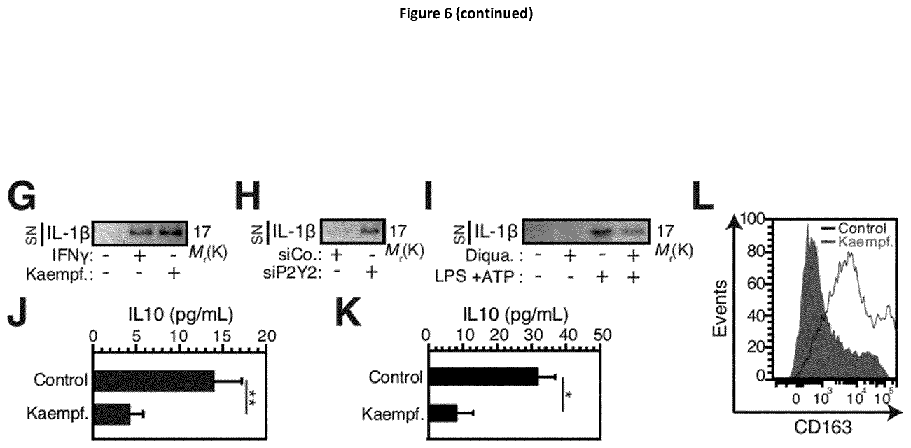

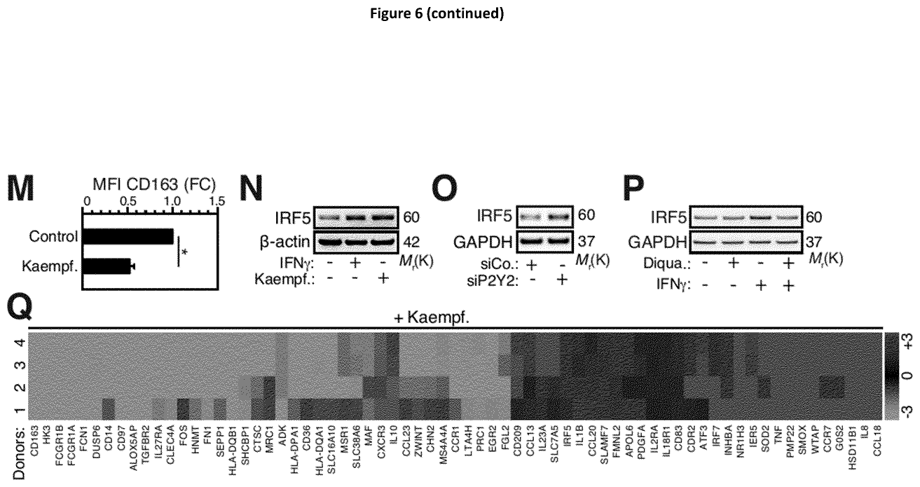

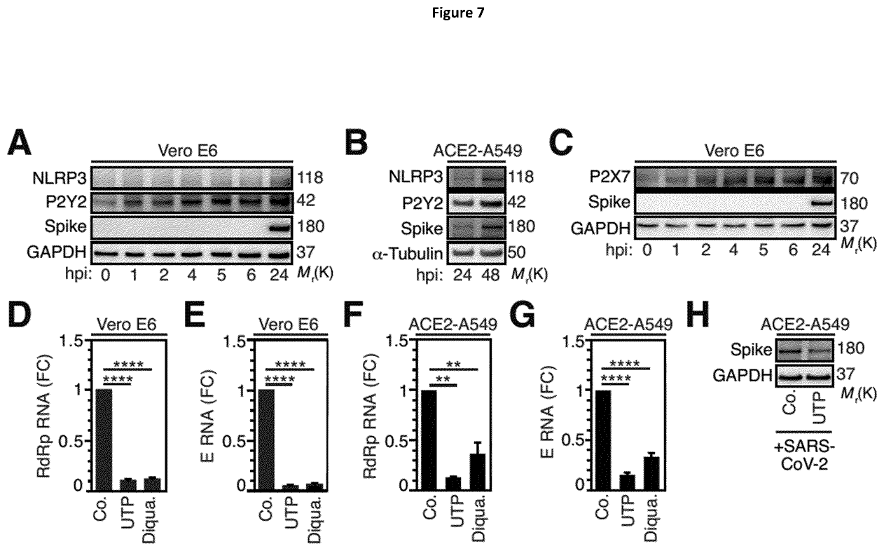

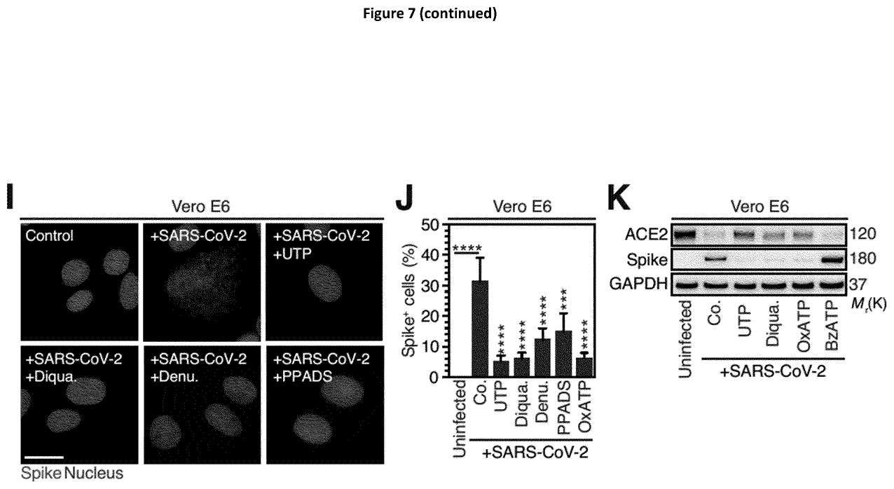

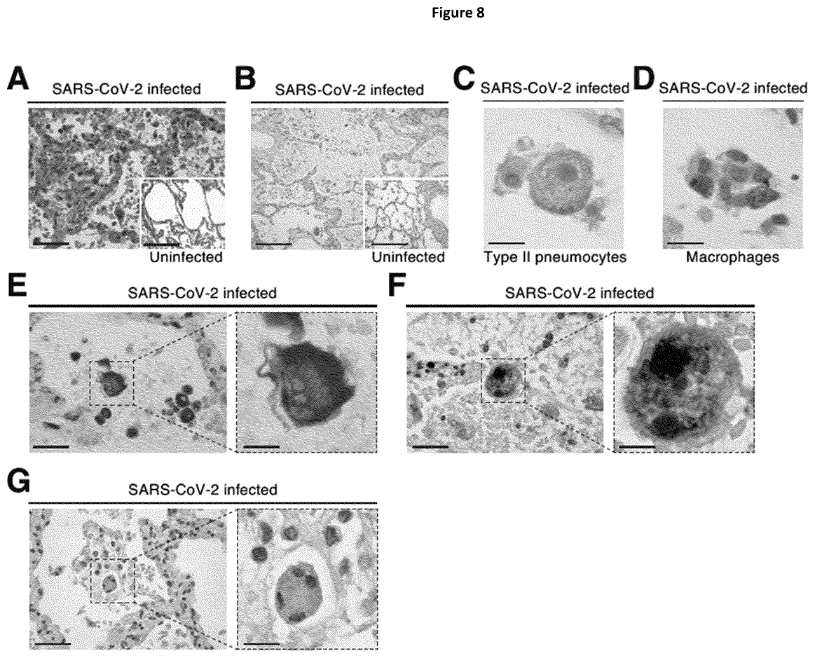

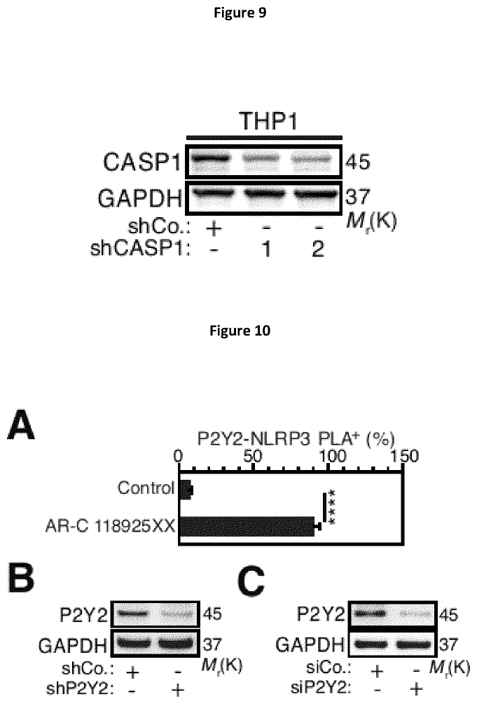

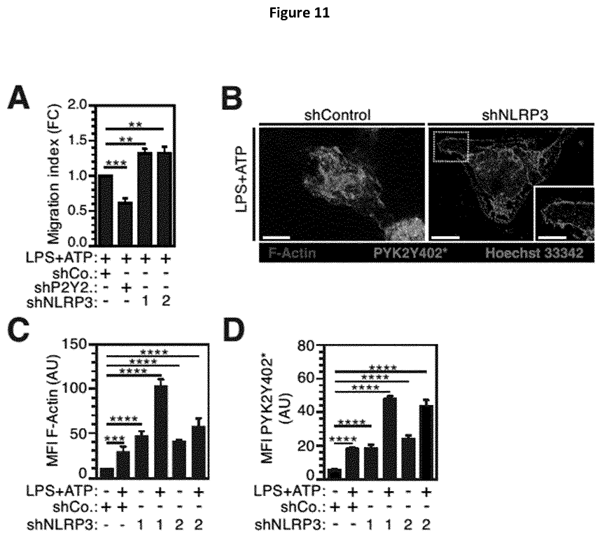

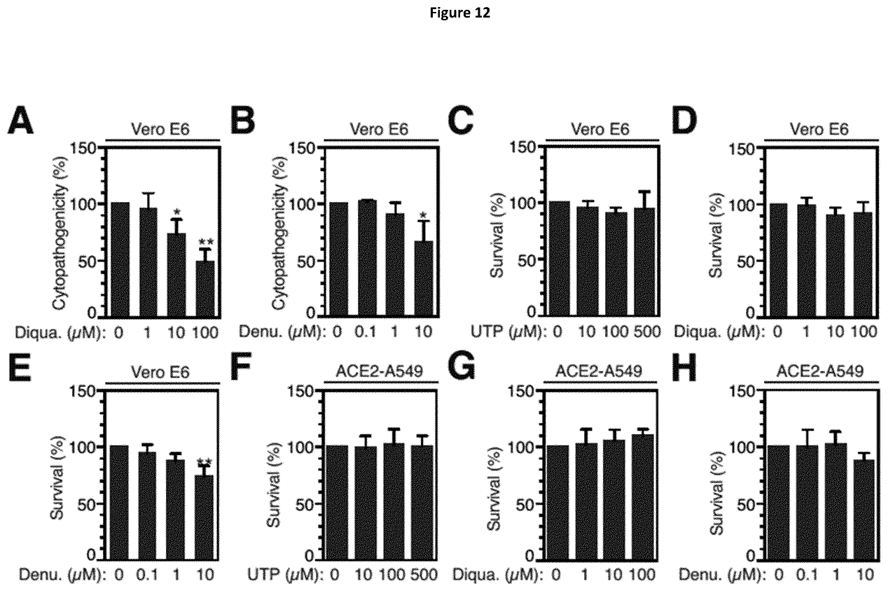

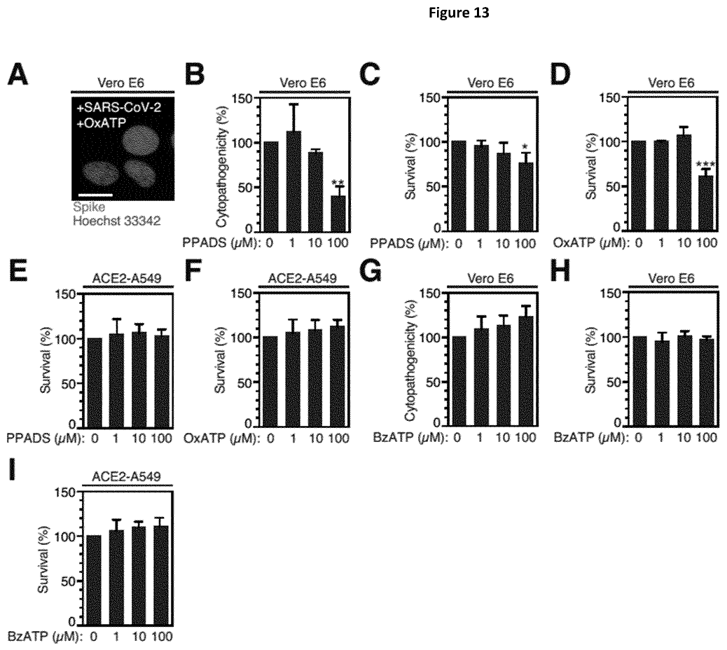

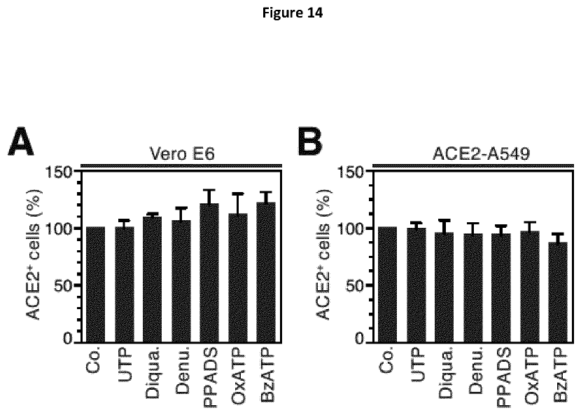

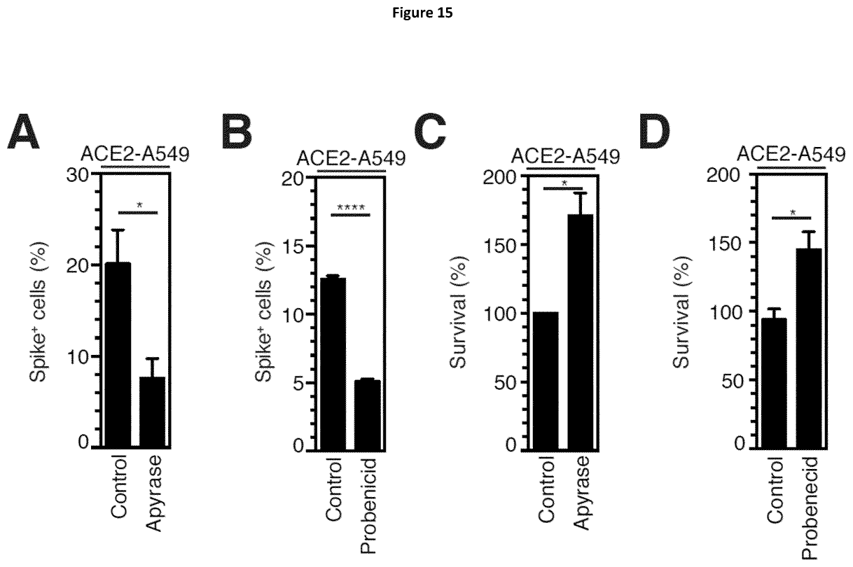

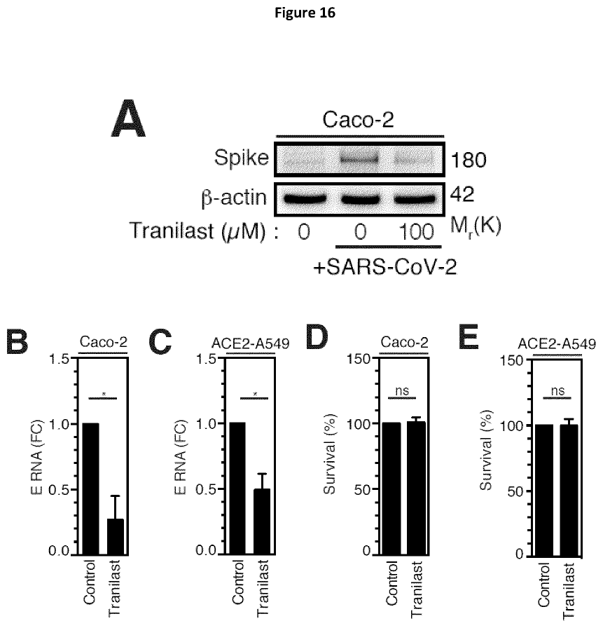

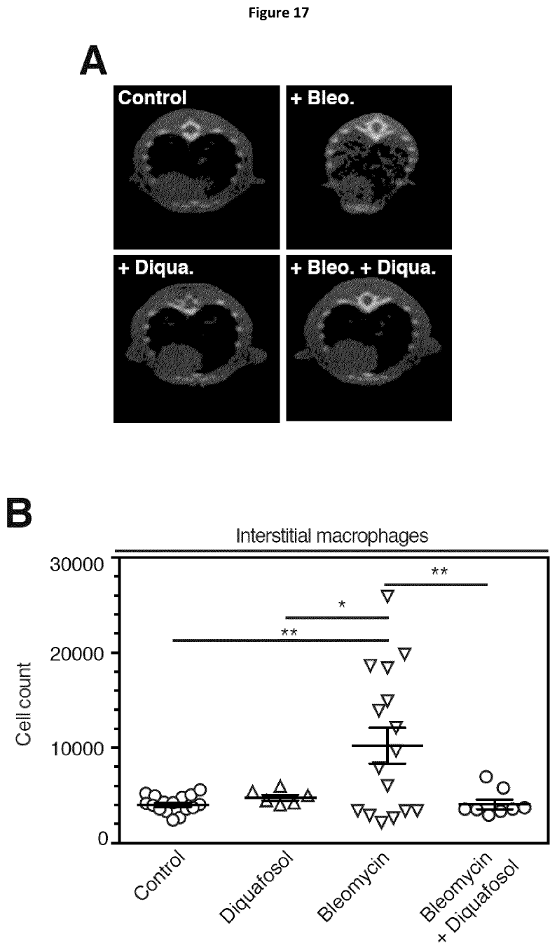

In parallel, the impact of purinergic modulators on cell damages elicited by SARS-CoV-2 infection was evaluated. Vero cells were infected with SARS-COV-2 during 72 hours in presence of indicated concentrations of UTP (FIG. 3A), Diquafosol (FIG. 3B), Denufosol (FIG. 3C) and BzATP (FIG. 3D). P2Y2 agonists exhibited inhibitory effects on cytopathogenicity (FIG. 3A-3C) and conversely as expected, BzATP seemed to stimulate the cytopathogenic effects associated with SARS-CoV-2 infection (FIG. 2D).

These results indicate that the modulation of purinergic receptor activity during SARS-CoV-2 infection affects SARS-CoV-2 pathogenicity. Altogether, these results demonstrate that purinergic receptors and extracellular nucleotides play a central rote during SARS-CoV-2 infection.

### 2.3. Mechanisms of Action of the Modulators of the Invention

The rapid worldwide spread of severe acute respiratory syndrome coronavirus 2 (SARS-CoV-2) poses a global health emergency that has not been resolved. Even though over 33,282,969 people are infected and more than 1,000,867 people have died from SARS-CoV-2 infection (https://coronavirus.jhu.edu/map.html), no cure or prophylactic treatment is currently available. With the exception of the antiviral remdesivir, which was authorized by the US Food and Drug administration for emergency use for the treatment of coronavirus 19 (COVID-19) (https://www.fda.gov), and steroid treatment which partially reduces the mortality of COVID-19 patients with severe disease (1), the majority of preclinical studies and clinical trials evaluating the repurposing of anti-malarial compounds (such as hydroxychloroquine and chloroquine), nonspecific anti-viral agents (such as lopinavir and ritonavir) or anti-inflammatory drugs (such as the IL-6 receptor antagonist tocilizumab) for the treatment of COVID-19 failed to identify highly effective treatment. A better understanding of mechanisms whereby SARS-COV-2 hijacks host cells for viral entry and dysregulates the anti-viral immune response is urgently needed to develop efficient therapeutic strategies for COVID-19.

The entry of SARS-CoV-2 into host cells starts with binding of viral spike (S) glycoprotein to angiotensin converting enzyme 2 (ACE-2) and with subsequent priming of S glycoprotein by the serine protease TMPRSS2 or cathepsin B/L (2, 3). The viral membrane then fuses with host cellular membranes (2, 4), leading to the release of viral RNA into host cytosol and replication using specialized proteins (such as RNA-dependent RNA polymerase (RdRp) (5)), intracellular expression of viral structural proteins (such as E and S proteins) and finally to the assembly and release of viral progeny (6). Host factors (such as p38MAPK, CK2, AXL and kinase PIFFWE kinases) are involved in the regulation of early and late steps of SARS-CoV-2 infection (7), but the host cellular pathways used by SARS-COV-2 to establish a viral infection are still poorly understood. Even though SARS-CoV-2-infected people are mainly asymptomatic or exhibit mild to moderate symptoms, approximately 15% of patients experience severe disease with atypical pneumonia and 5% develop an acute respiratory distress syndrome (ARDS) and/or multiple organ failure that is associated with a high mortality rate (around 50%) (8). Studies of COVID-19 patients with severe disease revealed a high level of plasma pro-inflammatory cytokines (including IL-1β, IL-6, IL-10, IL-18 and TNF) (9) and lactate dehydrogenase (LDH) (10), indicating overt hyper-inflammation during COVID-19, also known as “cytokine storm” or “cytokine release syndrome”. Until now, few molecular mechanisms driving COVID-19 associated hyper-inflammation have been identified and proposed to explain the pathogenesis of COVID-19.

Nucleotide-binding domain leucine-rich repeat-containing receptor (NLR) proteins and purinergic (P2) receptors are the main germline-encoded pattern recognition receptors regulating the secretion of IL-1 family members in response to microbial infection, inflammation, and inflammatory diseases. Upon activation, NLR protein 3 (NLRP3), which is the most studied NLR protein (11), forms large complexes, called inflammasomes, which activate caspase-1, induce the release of mature cytokines IL-1β and IL-18 (11, 12) and can lead to the inflammatory cell death of stimulated, stressed or infected host cells, which is also known as pyroptosis (13), SARS-CoV-2 viral proteins such as viral spike (S) glycoprotein (14), SARS-Cov open reading frame-8b (15) and the transmembrane pore-forming viral Viroporin 3a (also known as SARS-COV 3a) (16) were recently shown to activate the NLRP3 inflammasome, thus indicating that the NLRP3 inflammasome could represent a novel molecular target for the treatment of COVID-19. Purinergic receptors are membrane-bound innate receptors that bind extracellular nucleotides (such as adenosine triphosphate (ATP) and uridine triphosphate (UTP)), and control numerous cellular functions (such as cytokine secretion and migration) mainly on immune cells, but also on other cell types that are involved in SARS-CoV-2 pathogenesis such as type 1 and 2 pneumocytes, endothelial cells, platelets, cardiomyocytes and kidney cells (17, 18).

Purinergic receptors are divided into two families, the tonotropic P2X receptors and the metabotropic P2Y receptors, which can regulate the NLRP3 inflammasome (18-20). P2X7 activation was extensively shown to control NLRP3 inflammasome activation and cytokine release in response to danger signals (21). We recently demonstrated that the purinergic receptor P2Y2 interacts with NLRP3 and induces its ubiquitination and degradation (20), indicating that P2Y2 may potentially regulate negatively NLRP3 inflammasome activation. We previously revealed that purinergic receptors P2Y2 and P2X7 also control viral entry through the modulation of the fusogenic activity of HIV-1 envelope (22). The contribution of purinergic receptors to viral infection has been confirmed with other purinergic receptors and with several viruses (such as human cytomegalovirus and hepatitis B, C and D viruses) (23). Recently, we demonstrated that NLRP3 represses P2Y2-dependent viral entry during the early steps of HIV-1 infection (20). In addition, the P2Y2-NLRP3 interaction is enhanced during the inflammation, which is associated with chronic infections with HIV-1 and SIV (20). In this context, we hypothesized that the P2Y2-NLRP3 interaction could be enhanced during COVID-19 and that the therapeutic modulation of P2Y2 and NLRP3 could regulate both susceptibility of host cells to SARS-CoV-2 infection and hyper-inflammation associated with COVID-19. The contribution of P2X7 to SARS-CoV-2 pathogenesis was also addressed in this study.

### 2.3.1. Material and Methods

### 2.3.1.1. Viruses, Cells and Chemical Compounds

The BetaCoV/France/IDF0372/2020 SARS-CoV-2 strain was provided by Dr. Benoit Visseaux from the group of Prof. Diane Descamps (UMR 5 1135, Hôpital Bichat, Paris) and by the National Reference Center For Respiratory Viruses (Institut Pasteur, Paris, France). For in vitro studies, viral stocks were prepared by propagation in African green monkey kidney epithelial (Vero E6) cells in a biosafety level-3 (BLS-3) laboratory and titrated using lysis plaque assay as previously described (53). SARS-CoV-2 stock titer was 2×10⁶ PFU/mL. The supernatant was aliquoted and stored at −80° C. For in vivo studies, hCOV-19/France/IDF0372/2020 strain was amplified and virus stock produced as previously-described (54). Vero E6 cells were purchased from ATCC (ATCC CRL-1587) and cultured in Dulbecco's modified Eagle's medium (DMEM, Gibco, USA) with 10% heat inactivated fetal bovine serum (FBS), 100 UI/mL penicillin (Life technology), and 100 μg/mL streptomycin (Life technology) at 37° C. ACE2-overexpressing 1549 (ACE2-A549) cells were a gift from Dr. Olivier Schwartz (Institut Pasteur, Paris). Monocyte THP1 cells (ATCC TIB2002) were obtained from ATCC and were maintained in RMPI-1640-Glutamax medium supplemented with 10% heat inactivated FBS, 100 UI/mL penicillin, and 100 μg/mL streptomycin. Buffy coats from healthy donors were obtained from the French blood bank (Etablissement Français du Sang (EFS)). Informed written consent from each donor was obtained accordingly to French law. To generate Monocytes Derived Macrophages (MDMs), human monocytes were separated from peripheral blood mononuclear cells (PBMCs) by adherence to the plastic, detached and cultured for 6 days in hydrophobic Teflon dishes (Lumox Duthsher) in macrophage medium (RPMI 1640 supplemented with 200 mM L-glutamine, 100 UI of penicillin, 100 μg streptomycin, 10 mM HEPES, 10 mM sodium pyruvate, 50 μM β-mercaptoethanol, 1% minimum essential medium vitamins, 1% non-essential amino acids (Life technology)) supplemented with 15% of heat inactivated human serum AB (Life technology). Then, monocytes-derived macrophages (MDMs) were harvested and resuspended in macrophage medium containing 10% of FBS, as previously described (20, 55 , 56). Control THP1 cells (sh.Co.) and THP1 cells depleted for NLRP3 (sh-1NLRP3 and sh-2NLRP3) or P2Y2 (shP2Y2) were previously published (20). THP1 cells depleted for CASP1 (sh-1CASP1 and sh-2CASP1) were produced following the same experimental procedure and simultaneously to control and NLRP3- or P2Y2-depleted THP1 cells as indicated below. Uridine 5′-triphosphate (UTP) (#U6625), 2′(3′)-O-(4-Benzoylbenzoyl) adenosine 5′-triphosphate (BzATP) (#B6396), Kaempferol (#K0133), Suramin (#S2671), oxidized ATP (OxATP) (#A6779), pyridoxal-phosphate-6-azopheny-2′,4′-disulfonate (PPADS) (#P178) were purchased from Sigma-Aldrich. AR-C118925XX (#4890), Diquafosol (#HY-B0606) and Denufosol (#ND45968) were respectively from Tocris, MedChemExpress and Biosynth Carbosynth. Phorbol myristate acetate (PMA) (#tlrl-pma) was from Invivogen and recombinant human interferon g (IFNγ) was from R&D systems (#285-IF).

### 2.3.1.2. Patients' Samples

This non-interventional study received approval by the institutional review boards of Gustave Roussy hospital (Villejuif, France) and the National Institute for Infectious Disease <<Lazzaro Spallanzani>> and following the principles stated in the Declaration of Helsinski. Peripheral blood samples (Gustave Roussy) were obtained with informed consent of patients. The collection of blood samples from SARS-CoV-2 (n=7) and positive (n-18) patients was obtained from patients that were detected either negative or positive for SARS-CoV-2 by real time PCR (RT-PCR) at days before and at the same day of the blood sampling. The SARS-CoV-2 detection was performed on nasopharyngeal samples by RT-PCR using the GeneFinder COVID-19 PLUS RealAmp Kit (ELITECH), which detects SARS-CoV-2 by negative amplification of RdRp gene, E gene and N gene according to WHO recommended protocol. Moderate SARS-CoV-2 cases were defined as WHO Ordinal Scale for Clinical Improvement (OSCI) scale 3 and 4 and ≤5L/min of oxygen flow to maintain oxygen saturation (SpO2)>94%. Severe SARS-CoV-2 cases were defined as OCSI scale 4-8 and prolonged need of a ≥6 L/min of oxygen flow to maintain SpO2>94%. In this study all the five severe SARS-CoV-2 patients were admitted to the intensive care unit and needed mechanical ventilation. Fresh blood samples (250 μl) of SARS-CoV2 positive (n=18) or negative (n=7) patients were dropped on the microscopy slides that were pre-treated with Poly-L-lysine solution (0.1%) to let peripheral blood cells adhere for 1 hour at room temperature. Adherent cells were then washed with phosphate buffered saline (PBS) solution, dried for 5 minutes and fixed by 2% paraformaldehyde (PFA) during 20 minutes at room temperature. After washing with PBS, the slides were conserved at 4° C. in PBS. Human autopsies were performed at the National Institute for Infectious Diseases Lazzaro Spallanzani-IRCCS Hospital (Rome, Italy) according to guidance for post-mortem collection and submission of specimens and biosafety practices (CDC March 2020, Interim Guidance and (57)) to reduce the risk of transmission of infectious pathogens during and after the post-mortem examination. Autopsies were performed in accordance with the law owing to the unknown cause of death, and to both scientific and public interest in a pandemic novel disease. All performed procedures and investigations were in accordance with the ethical standards of the Institutional Review Board of Lazzaro Spallanzani National Institute for Infectious Disease (Ethics Committee Approval number: n° 9/2020). Post-mortem lung sections were obtained from 3 non-COVID-19 patients and 7 COVID-19 patients with severe disease. Controls (n=3) were patients deceased after hemorragia, cardiorespiratory failure or interstitial pneumonia associated with pulmonary capillaritis. All COVID-19 patients (n=7) deceased after cardiorespiratory failure and exhibited in their vast majority diffuse alveolar damage (n=6) or fibrosis (n=1). Detection of SARS-CoV-2 was performed by RT-PCR on all patients using ocular, nasopharyngeal, oropharyngeal and rectal swabs.

2.3.1.3. Samples from Non-Human Primates

Cynomolgus macaques (Macaca fascicularis) aged from 4 to 7 years originating from Mauritius Island and housed in Infectious Disease Models for Innovative Therapies (IDMIT) infrastructure of Commissariat à l'Energie Atomique et aux Energies Alternatives (CEA, Fontenay-aux-roses, France) were used. The protocols were approved by the ethical committee of animal experimentations of CEA under the protocol number CEA #44. Challenged animals were exposed to a total dose of 10⁶ PFU of SARS-CoV-2 (BetaCoV/France/IDF0372/2020 SARS-CoV-2 strain) via the combination of intranasal and intra-tracheal routes. The bronchoalveolar lavage fluids (BALFs) from SARS-CoV2 negative (n=4) and positive (n=5) Cynomoglus macaques were obtained three days after infections. Viral loads (0.76-2.4 (copies/mL)) were assessed in bronchoalveolar lavages by RT-PCR with a plasmid standard concentration range containing an RdRp gene fragment including the RdRp-IP4 RT-PCR target sequence. The protocol describing the procedure for the detection of SARS-CoV-2 is available on the WHO website (https://www.who.int/docs/default-source/coronaviruse/real-time-rt-pcr-assays-for-the-detection-of-sars-cov-2-institut-pasteur-paris. pdf?sfvrsn=3662fcb6_2). All BALFs were chilled in the ice up during handling, well mixed by low vortexing and filtered using cell strainers of 100 μm pore membranes (Falcon #352360) to eliminate debris. Cellular suspension was then centrifuged at 1000 rpm for 10 minutes at 4 ° C. and washed twice with 50 mL cold PBS containing 20% FBS. Viable cells (0.5×10⁶) were suspended in 200-μl cold PBS containing 20% FBS and dropped on poly-L-Lysine coated slides by using cytospin centrifuge (Cytospin 2 Shandon, Block scientific) at 800 rpm for 3 minutes. BALF cells were then air dried on slides for 30 minutes, fixed with 4% PFA solution for 20 minutes, washed twice with PBS and conserved at 4° C.

2.3.1.4. Samples from P2y2^{+/+} and P2y2^{-/-} Mice

Two weeks old P2y2^{+/+} and P2y2^{-/-} transgenic mice were obtained from Dr. Isabelle Couillin (58) and sacrificed upon arrival following the Federation of European Laboratory Animal Science Association guidelines and in accordance with the Ethical Committee of the Gustave Roussy Cancer Campus (CEE A26) (Villejuif, France). After sacrifice, plasmatic serum was aliquoted and stored at −80° C., lung biopsies were either fixed or digested for biological analysis as previously described (59).

2.3.1.5. In vitro infection with SARS-CoV-2

Vero (E6) cells and ACE2-4549 cells were infected during indicated times with SARS-CoV-2 (BetaCoV/France/IDF0372/2020) at multiplicity of infection (MOI) from 0.1 to 0.2, for infectivity analysis and from MOI 1 to 2 for cytopathogenicity analyses, in absence or in presence of 500 μM UTP, 100 μM Diquafosol, 5 μM Denufosol, 50 μM PPADS, 100 μM OxATP or 100 μM BzATP unless stated otherwise.

### 2.3.1.6. RNA Interference

Plasmids coding for the gag-pot HIV-1 genes (pCMV GAG-POL HIV University of Michigan), for the vector genome carrying shRNA of interest (pLKO.1 shRNA, Thermo Scientific) and for the plasmid coding for an envelope of VSVG (pMDG-VSV-G) were transfected into HEK293T cells using calcium phosphate reagent (Promega) to obtain lentiviral vector particles. After 48 hours, supernatants were filtered using 0.45-μm cellulose acetate filters (Sartorius stedim), aliquoted and stored at −80° C. Monocytic THP1 cells (4×10⁶) were then transduced during 24 hours and grown in medium containing 1 μg/mL puromycin (Invivogen). Control THP1 (PLKO.1) cells and P2Y2- (shP2Y2), NLRP3- (sh-1NLRP3 and sh-2NLRP3) and CASP1- (sh-1CASP1 and sh-2CASP1)-depleted THP1 cells were thus obtained. Validation of shCo., shP2Y2, sh-1NLRP3, sh-2NLRP3-containing THP1 cells were previously described and validated (20). MDMs were transfected using smart pools of siGenome non-targeting control and P2Y2 specific siRNAs from Dharmacon as previously described (20, 55, 56). After 48 hours of transfection, cell lysates and supernatants were analyzed for protein expression by western blot, flow cytometry and ELISA. Sequences of Human siRNA, siGENOME 5MARTpool and shRNA used in this study are shown in the following table:

| | | | |
|---|---|---|---|
| Human target | sg | Target sequence | SEQ ID NO |

siGENOME Non Targeting

| | | | |
|---|---|---|---|
| siRNA SMARTpool | 1 | UAGCGACUAAACACAUCAA | SEQ ID NO: 9 |
| | 2 | UAAGGCUAUGAAGAGAUAC | SEQ ID NO: 10 |
| | 3 | AUGUAUUGGCCUGUAUUAG | SEQ ID NO: 11 |
| | 4 | AUGAACGUGAAUUGCUCAA | SEQ ID NO: 12 |

siGENOME SMARTpool

| | | | |
|---|---|---|---|
| P2Y2 | 1 | UGCCUAGGGCCAAGCGCAA | SEQ ID NO: 13 |
| | 2 | UAACUGGAGCUCCGAUUUA | SEQ ID NO: 14 |
| | 3 | UCUCAGGAGUAGUCUCAUA | SEQ ID NO: 15 |
| | 4 | AGUCAUCGUUUGUGUGUAU | SEQ ID NO: 16 |
| pLKO.1 shP2Y2 | 1 | ATGTTCCACCTGGCTGTGT | SEQ ID NO: 17 |
| | | CTGATGCACT | |
| pLKO.1 | 1 | ATAATGAGAGCAAGACGTG | SEQ ID NO: 18 |
| sh-1Caspase-1 | | TG | |
| pLKO.1 | 2 | AGCATCATCCTCAAACTCT | SEQ ID NO: 19 |
| sh-2Caspase-1 | | TC | |

### 2.3.1.7. Quantitative RT-PCR for SARS-CoV-2

After infection, total cell RNA was extracted using QIAshredder kit (#79654, Qiagen) and the RNeasy kit plus Mini kit (#74134, Qiagen) according to the manufacturer's instructions. RNA was recovered according to the manufacturer's instructions for total cell RNA extraction. Quantifications were performed by real-time PCR on a Light Cycler instrument (Roche Diagnostics, Meylan, France) using the second-derivative-maximum method provided by the Light Cycler quantification software (version 3.5 (Roche Diagnostics)). Standard curves for SARS-CoV-2 RNA quantifications were provided in the quantification kit and were generated by amplification of serial dilutions of the provided positive control. Sequences of the oligonucleotides and probes used to quantify the RdRp and E genes are given in the following Table.

| Gene | Primers and Probe sequences (SEQ ID NO: |
|---|---|
| target | 3-5 and 6-8) |
| SARS- | RdRp SARSr-F 5′: gTgARATggTCATgTgTggCggRdRp |
| Cov-2 | RdRp SARSr-R 5′: CARATgTTAAASACACTATTAgCATA |
| RdRp | SARSr-P2 5′: |
| | 6FAMCAggTggAACCTCATCAggAgATgC-BBQ |
| SARS- | E Sarbeco F 5′: ACAggTACgTTAATAgTTAATAgCgT |
| Cov-2 | E Sarbeco R 5′: ATATTgCAgCAgTACgCACACA. |
| E-gene | E Sarbeco P1 5′: 6FAM- |
| | CAggTggAACCTCATCAggAgATgC-BBQ |

The amplification of RdRp and E genes was obtained after 5 minutes of reverse transcription, 5 minutes of denaturation and 45 cycles with the following steps (95° C. during 5 seconds, 60° C. during 15 seconds and 72° C. during 15 seconds). Results were normalized by the total amount of RNA in the sample and also reported to the condition without any compound (control condition). Data are presented as fold changes and were calculated with relative quantification of ΔΔCT obtained from quantitative RT-PCR.

### 2.3.1.8. Immunofluorescence and Flow Cytometry

Peripheral human blood cells, non-human primates cells, ACE2-A549 and THP1 cells were permeabilized with 0.3% Triton-X100, blocked with 10% FBS for 1 hour at room temperature before overnight staining at 4° C. with anti-NLRP3 (#ab4207, Abcam) and anti-P2Y2 (#APR-010, Alomone) antibodies at 1/50 dilutions. After washings with PBS, the proximity ligation assay was performed according to manufacture's instructions. Briefly, the primary antibodies were hybridized with the Duolink In Situ PLA Probes anti Rabbit PLUS (#DU092002, Sigma) and anti-Goat Minus PLUS (#DU092006, Sigma) for 1 hour at 37° C. followed by a ligation step of 30 minutes at 37° C. and an amplification step of 1 hour and 40 minutes at 37° C. The ligase and the polymerase enzymes catalyzing these reactions were included in the Duolink® In Situ Detection Reagents Green (#DUO92014, Sigma). Additional immunostaining step was done at room temperature in humid chamber. For THP1 and ACE2-A549 cells, nuclei were stained with Hoechst 33342 (#1874027, Invitrogen) (1/1000) for 30 minutes. Peripheral human blood cells were incubated with Alexa Fluor 647 anti-CD3 (#300416, BioLegend) (1/50) and Alexa Fluor 594 anti-human CD14 (#325630, BioLegend) (1/50) and Hoechst 33342 (1/500) for 2 hours. Bronchoalveolar lavage fluid (BALF) cells obtained from non-human primates were incubated with Alexa Fluor 647 anti-human CD68 (#562111, BD Pharmigen) (1/50) for two hours. After washings with PBS, cells were air dried at room temperature for 1 hour on slides and protected from the light. Then, slides were mounted with Duolink In situ Mounting Medium with DAPI (#DU082040, Sigma) and conserved at 4° C. For the detection of SARS-CoV-2-infected cells in vitro, Vero E6 and ACE2-A549 cells were fixed in 4% paraformaldehyde in PBS for 5 minutes, permeabilized with 0.3% Triton (Sigma) in PBS, and incubated in PBS containing 10% FBS for 1 hour. SARS-CoV-2-infected ACE2-A549 cells were subjected to P2Y2-NLRP3 PLA staining as described above, but SARS-CoV-2-infected Vero E6 and ACE2-4549 cells also were incubated during 2 hours with mouse anti-Spike S antibody (#GTX632604, Genetex) and then during 1 hour with goat anti-mouse IgG conjugated to Alexa Fluor 546 (#A11030, Invitrogen). Nuclei were also stained with Hoechst 33342 (#H3570, Invitrogen) as previously described. PMA-differentiated THP1 macrophages and MDMs that were treated during 72 hours with 100 or 1000 ng/mL IFNγ or 10 ng/mL LPS were also analyzed for P2Y2-NLRP3 PLA staining following the same procedure. The visualization and quantification of PLA assays were performed in blind with confocal microscopy (SP8, Leica), which is equipped with two PMT and two high sensitivity hybrid detectors using a 63× oil objective. The representative PLA cells were imaged by confocal microscopy (SP8, Leica) using hybrid detectors (pinhole airy: 1; pixel size: 180 nm, magnification zoom: 3.5×) at optimal optical sectioning (OOS) of 0.8 μm. The confocal PLA images were then analyzed by Image J software in the best focal plan for the construction of z projection images on maximum intensity. LPS+ATP-stimulated, PMA-THP1 macrophages that were depleted (or not) for NLRP3 were analyzed for PYK2Y402* (#3291, Cell Signaling) and F-actin polymerization (using Alexa Fluor 488 Phalloïdin (#A12379, Invitrogen)) by confocal microscopy as previously published (20). For immunofluorescence analysis of mouse lungs, 4-82 m sections were cut from the paraffin blocks of the paraformaldehyde fixed tissues from mice. After paraffin removal, slides were subjected to antigen retrieval by microwave boiling in 1 mmol/L EDTA pH 9.0. After permeabilization with 0,3% Triton during 5 minutes and saturation in PBS containing 20% FBS during 1 hour, slides were first stained with green TUNEL assay (#Roche, #11684809910) during 1 hour at 37° C. according to the manufacturer's instructions and then incubated with anti-CD40 (#14-0401, ebioscience) or anti-CASP1 p10 (#sc-22164, Santa Cruz) overnight at 4° C. Then, cells were incubated with anti-rat IgG conjugated to Alexa Fluor 546 (#A11081, Invitrogen) or anti-goat IgG conjugated to Alexa Fluor 647 (#A21447, Invitrogen) fluorochromes at room temperature during 1 hour and 30 minutes. Cells were analyzed by fluorescent confocal microscopy on Leica SPE (using a 63× objective). Z series of optical sections at 0.4-μm increments were acquired. For flow-cytometry analysis, MDMs (10⁶ cells/mL) were harvested after indicated treatments in RPM) complete medium, washed twice with PBS, saturated at 4° C. for 20 minutes in PBS containing 10% FBS and incubated with anti-CD163 Alexa Fluor 647 (#562669, B D Pharmingen)) antibodies during 1 hour and 30 minutes. Membrane expression of CD163 was then analyzed using LSRFortessa (B D) flow cytometer. ACE2 positive Vero and ACE2-A549 cells were determined after two hours blocking with 20% FBS and overnight incubation with anti-Angiotensin converting enzyme (CD143) (#557928, B D Biosciences) for at 4° C. Mice alveolar macrophages were dissociated from lung (using Lung Dissociation kit from Miltenyi Biotech) and analyzed using anti-CD11b (APC-Cy7) (#557657, B D Pharmingen), anti-CD11c (PE-Cy7) (#117318, BioLegend), anti-CD40 (eFluo710) (#46-0401, ebioscience), anti-F4/80 (RTC) (#11-4801, ebioscience) and anti-Ly-6G (GR-1-PE) (#12-5931, ebioscience) using LSRFortessa (BD) flow cytometer as previously reported (59).

### 2.3.1.9. Western Blots

Total cellular proteins were extracted and lysed in appropriated buffer containing 250 mM NaCl, 0.1% NP-40, 5 mM EDTA. 10 mM Na3VO4, 10 mM NaF, 5 mM DTT, 3 mM Na4P2O7, 1 mM EGTA, 10 mM Glycerol phosphate, 10 mM Tris-HCl (pH=7.5) and the protease and phosphatase inhibitors (Roche)). Then, 10-40 μg of protein extracts were run on 4-12% or 12% SDS-PAGE and transferred at 4° C. onto a nitrocellulose membrane (0.2 Micron). After 2 hours saturation at room temperature with 5% nonfat milk or BSA (Bovine Serum Albumin) in Tris-buffered saline and 0.1% Tween 20 (TBS-Tween), nitrocellulose membranes were incubated with primary antibody at 4° C. overnight. Horseradish peroxidase (HRP)-conjugated goat anti-mouse or anti-rabbit antibodies (SouthernBiotech) or rabbit anti-goat antibodies (SouthernBiotech) were then incubated for 1 hour and revealed with the enhanced ECL detection system (GE Healthcare). The primary antibodies against P2Y2 (#APR-010) and P2X7 (#APR-004) were obtained from Alomone laboratories. Primary antibodies against ACE2 (#AF933) and Spike S (#GTX632604) were from Biotechene and GeneTex. Primary antibodies against IL-1β (#ab2105), IRF5 (#ab21689) and β-Actin-HRP (#ab49900) were purchased from Abcam. Anti-NLRP3 (Cryo-2), anti-CASP1 (#2225), anti-α-Tubulin (#T9026) and GAPDH (#MAB374) were from Adipogen, Cell signaling, Sigma and Millipore, respectively.

### 2.3.1.10. Detection of Cytokines

Plasma serum from P2y2^{+/+} and P2y2^{-/-} mice and supernatants harvested from PMA-stimulated THP1 macrophages that were depleted or not for P2Y2 or NLRP3, and/or stimulated with 10 ng/mL LPS during 3 hours and 5 mM ATP during 6 hours or with 20 ng/mL IFNγ, in presence or in absence of 100 μM Suramin, 100 μM OxATP, 100 μM Kaempferol or 100 μM Diquafosol at indicated times were analyzed using western blot or ELISA for IL-1β (Ebioscience) and IL-10 (BD) according to the manufacturers' instructions.

### 2.3.1.11. Cell Viability Assays

Human THP1 cells were cultured in RPMI 1640 media, supplemented with 10% FBS. THP1 cells were differentiated by treatment for 3 hours with 100 nM phorbol-12-myristate-13-acetate (PMA, Invivogen). After 2 days, control or THP1 cells depleted or not for P2Y2, NLRP3 or CASP1 were stimulated for 3 hours with ultrapure LPS from E. coli (10 ng/mL, Sigma) and for 6 hours with ATP (5 mM, Sigma), and analyzed for LDH release using LDH kit (Roche). Cell viability in drug-treated cells was also measured. Vero E6 cells and ACE2-A549 cells were pretreated with indicated concentrations of UTP, Diquafosol, Denufosol, PPADS, OxATP and BzATP during 4 hours before infection and infected or not with SARS-CoV-2 BetaCoV/France/IDF0372/2020 strain with a multiplicity of infection between 1 and 2. Cell viability was determined after 72 hours using 3-(4,5-dimethylthiazol-2-yl)-2,5-diphenyltetrazolium bromide (MTT) assay (#M5655, Sigma) following manufacturer's instructions. Cell viability was also performed in uninfected Vero E6 cells and ACE2-A549 cells with the same compound dilutions.

### 2.3.1.12. Macrophage Migration Assay

The migration of treated-PMA-primed THP1 cells was determined using a Boyden chamber system (Roche CIM16 plate XCELLigence DP) 9 hours after LPS (10 ng/mL) and ATP (5 mM) stimulation.

### 2.3.1.13. Microarray Assay

MDMS were treated with 100 μM Kaempferol during 72 hours. Then, mRNAs were isolated using RNeasy kit (#74104, Quiagen) and gene expression analyses were performed with Agilent® SurePrint G3 Human GE 8x60K Microarray (Agilent Technologies, AMADID 39494) with the following single-color design. RNAs were labeled with Cy3 using the one-color Agilent labeling kit (Low Input Quick Amp Labeling Kit 5190-2306) adapted for small amount of total RNA (100 ng total RNA per reaction). Hybridization were then performed on microarray using 800 ng of linearly amplified cRNA labeled, following the manufacturer protocol (Agilent SureHyb Chamber; 800 ng of labeled extract; duration of hybridization of 17 hours; 40 μL per array; Temperature of 65° C.). After washing in acetonitrile, slides were scanned by using an Agilent G2565 C DNA microarray scanner with defaults parameters (100° PMT, 3 μm resolution, at 20° C. in free ozone concentration environment). Microarray images were analyzed by means of Feature Extraction software version (10.7.3.1) from Agitent technologies. Defaults settings were used.

### 2.3.1.14. Microarray Data Processing and Analysis

Raw data files from Feature Extraction were imported into R with LIMMA (Smyth, 2004, Statistical applications in Genetics and molecular biology, vol 3, N°1, article 3), an R package from the Bioconductor project, and processed as follow: gMedianSignal data were imported, controls probes were systematically removed, and flagged probes (gisSaturated, glsFeatpopnOL, glsFeatNonUnifOL) were set to NA. Inter-array normalization was performed by quantile normalization. To get a single value for each transcript, taking the median of each replicated probes summarized data. Missing values were inferred using KNN algorithm from the package ‘impute’ from R bioconductor. Normalized data were then analyzed. To assess differentially expressed genes between two groups, we start by fitting a linear model to the data. Then, we used an empirical Bayes method to moderate the standard errors of the estimated log-fold changes. The top-ranked genes were selected with the following criteria: an absolute fold-change>2 and an adjusted p-value (FDR)<0.05.

### 2.3.1.15. Immunochemistry

Post-mortem lung specimens were fixed in formalin and embedded in paraffin. Tissue sections were deparaffinized, rehydrated, incubated in 10 mM sodium citrate, pH 6.0, microwaved for antigen retrieval and treated with 3% H2O2 to block endogenous peroxidase activity. Then, mouse antibodies against NLRP3/NALP3 (#AG-20B-0014, AdipoGen) (1:100), CD68 (#KP-1, Ventana) (prediluted), or double-stranded RNA (#J2-2004, Scicons J2) (1:500) and biotinylated goat anti-mouse IgG (#BA-9200, Vector) were incubated with lung sections. Immune-reactivities were vizualized using avidin-biotin complex-based peroxidase system (#PK-7100, Vector) and 3,3′-diaminobenzidine (DAB) peroxidase (HRP) substrate Kit (#SK-4100, Vector). Lung sections were also stained with hematoxylin and eosin, as previously described (60) and assessed by two independent observers without the knowledge of clinical diagnosis, using a Leica DM2500 LED Optical microscope and a 63× objective.

### 2,3.1.16. Statistical Analysis

For the pairwise comparison of two groups within the same cell line (THP1 monocytes, PMA-differentiated THP1 macrophages, Vero E6 or ACE2-A549), where the cell populations have normal distribution, we used the unpaired two-tailed t-test to compare the means±SEM (standard error of the mean). For the comparison of two groups of samples from independent individuals (blood cells of healthy donors or patients, bronchoalveolar lavage (BALF) cells of non-human primates and serum or organ cells of mice) where the sample populations did not assume the same distributions, we used the non-parametric unpaired two-tailed or one-tailed Mann-Whitney test to compare the means of the ranks of two groups. When the control sample values from independent individuals were normalized to the value of 1 to compare the fold changes in the treated group, we used the non-parametric test Wilcoxon matched-pairs signed rank test. For the multiple comparisons of more than two groups, we used the one-way ANOVA test when the sample groups have one variable parameter (such as gene knockdown or stimulus or drug treatments or infections). When the sample groups have two variable parameters (such as gene knockdowns and stimulus or drug treatments and/or infections) we used the two-way ANOVA test to compare the means of the absolute values, of the frequencies or of the fold changes. The p-values of the multiple comparisons of ANOVA tests were adjusted with the Tukey's Honestly Significant Difference (HSD) test when the means are compared with every other mean or with Dunnett's correction test when every mean is compared to the control mean. Statistical data were analyzed with Graphpad prism 6 and 8 software. Statistical significance was given as *P<0.05, **P<0.01, ***P<0.001, and ****P<0.0001.

### 2.3,2. Results

2.3.2.1. Expression of NLRP3 on Lung Tissue Samples Obtained from Uninfected and SARS-CoV-2-Infected Carriers To study the potential contribution of the P2Y2 and NLRP3 interaction to pathogenesis of SARS-CoV-2 infection, we first analyzed the expression of NLRP3 on lung tissue samples obtained from three uninfected carriers and seven SARS-CoV-2-infected carriers who died from COVID-19. Autopsies were stained with hematoxylin and eosin, or incubated with antibody against NLRP3 and analyzed. As recently described (24), parenchymal multifocal damages with intra-alveolar inflammation, fibrin and hyaline membrane formation that are consistent with a diagnosis of diffuse alveolar damage were observed in all SARS-CoV-2-infected carriers. The pattern of organizing pneumonia (with fibrotic organization and type 2 pneumocyte hyperplasia) and fibroblastic foci formed by loose organizing connective tissue consistent with alveolar duct fibrosis were also detected (FIG. 8A). Besides, the presence of inflammatory cells (composed mainly of macrophages and lymphocytes) was the main characteristic of COVID-19 patient autopsies. NLRP3 expression was then examined on these samples, and immuno-reactive NLRP3 was mainly detected in Type II pneumocytes (FIG. 8B and C) and alveolar macrophages (FIG. 8B and D) from both SARS-CoV-2 carriers and uninfected specimens, which have been previously shown to express P2Y2 (25, 26), indicating that these cellular targets of SARS-CoV-2 express both NLRP3 and P2Y2. Interestingly, we also detected in SARS-CoV-2 carriers, some syncytia that expressed macrophage marker, CD68 (FIG. 8E), NLRP3 (FIG. 8F) and viral RNA (FIG. 8G) in SARS-CoV-2 carriers, revealing a potential link between alveolar macrophages, NLRP3 expression and viral infection.
2.3.2.2. Modulation of the P2Y2-NLRP3 interaction in Peripheral Blood Cells During SARS-CoV-2 Infection.

Based on our previous work showing that the purinergic receptor P2Y2 interacts with and regulates NLRP3 protein expression during acute and chronic infection with HIV-1 (20), we then determined the ability of the P2Y2-NLRP3 interaction to be modulated in peripheral blood cells during SARS-CoV-2 infection. By means of a proximity ligation assay (PLA), we revealed that the P2Y2-NLRP3 interaction is enhanced in neutrophils (FIG. 4, A and B), monocytes (FIG. 4, C and D) and lymphocytes (FIG. 4, E and F) in the peripheral blood obtained from SARS-CoV-2 infected-patients, as compared with uninfected patients.

The enhancement of the P2Y2-NLRP3 interaction was also confirmed in macrophages when analyzing bronchoalveolar fluid lavages (BALFs) obtained from non-human primate (NHP) Macaca fascicularis that were infected with SARS-CoV-2, as compared with uninfected NHP (FIG. 4, G and H), demonstrating that the enhancement of P2Y2-NLRP3 interaction is also detected in alveolar macrophages which have been proposed to be key immune cells during ARDS. In addition, we observed an increased P2Y2-NLRP3 interaction in A549 type II pneumocytes that were infected during 48 hours with SARS-CoV-2 (FIG. 4, I and J). Altogether, these results indicate that the interaction between P2Y2 and NLRP3 is enhanced in type II pneumocytes during SARS-CoV-2 infection and in immune cells during COVID-19-associated hyper-inflammation, and emphasizes an unsuspected link between the purinergic receptor P2Y2, hyper-inflammation, and SARS-CoV-2 infection.

2.3.2.3. Expression of IL1β in P2y2^{-/ -} and P2y2^{+/ +} Mice

Since NLRP3 and P2Y2 have been separately reported to be involved in macrophage activation, which is a key event during SARS-CoV-2 pathogenesis (27-30), we then assessed basal expression levels of IL-1β in P2y2^{-/ -} and P2y2^{+/ +} mice. Interestingly, the baseline plasma levels of IL-1β in P2y2^{-/ -} mice were higher than in wild type controls (FIG. 5A), suggesting that P2Y2 acts to suppress production of IL-1β In addition, P2y2^{-/ -} mice exhibited a significant reduction in the frequency of alveolar macrophages carrying signs of pro-inflammatory activation (CD11⁺Gr1-F4/80⁺CD11c^{high}CD40^{high}) (FIG. 5, B and C). The vast majority of residual PP2y2^{-/ -} lung CD40⁺ macrophages contained cleaved caspase-1 and exhibited nuclear DNA fragmentation (as revealed by the TUNEL assay) (FIG. 5, D-G), suggesting that the pro-inflammatory P2y2^{-/ -} alveolar macrophages were eliminated through pyroptosis. Accordingly, PMA-differentiated THP1 macrophages released the cell death marker, lactate dehydrogenase (LDH), upon treatment or not with lipopolysaccharide (LPS) and ATP following P2Y2 inhibition with kaempferol (a specific P2Y2 inhibitor) (FIG. 5H). This LPS+ATP-induced THP1-cell death was inhibited by depletion of either NLRP3 (FIG. 5H) or CASP1 (FIG. 5H and FIG. 9). These results are consistent with the interpretation that P2Y2 inactivation favors NLRP3/CASP1-dependent macrophage pyroptosis and mirror the depletion of alveolar macrophages detected in COVID-19 patients with severe disease (31).

### 2.3.2.4. Modulation of the P2Y2-NRLP3 Interaction During Macrophage Activation

Given that macrophages are key cellular targets for the treatment of COVID-19 (30), we then analyzed the modulation of the P2Y2-NLRP3 interaction during macrophage activation. Interestingly, we found that the P2Y2-NLRP3 interaction is enhanced during pro-inflammatory macrophage reprogramming detected after treatment of PMA-THP1 macrophages (FIG. 6, A and C) and primary macrophages (FIGS. 6, B and D) with IFNγ (FIG. 6, A-D) and LPS (FIG. 6, A and C), as previously published (32). We observed that P2Y2 inhibition with a P2Y2 antagonist AR-C118925XX (FIG. 10A) also enhanced the P2Y2-NLRP3 interaction, indicating that the pharmacological inhibition of P2Y2 mimics the impact of the pro-inflammatory immune stimuli (such as LPS and IFNγ enhancement of NLRP3-P2Y2 interaction). We then simultaneously investigated the impact of NLRP3 or P2Y2 inactivation on various macrophage functions such as cytokine secretion and functional reprogramming. Pharmacological inhibition of P2Y2 with suramin (a non-specific P2X and P2Y antagonist) (FIG. 6E), with kaempferol (FIG. 6, E and G), or depletion of P2Y2 by means of specific short hairpin RNAs (FIG. 6F and 10B) or small interfering RNAs (FIG. 6H and 10C), increased IL-1β release from PMA-treated THP1 macrophages stimulated with LPS+ATP or from human monocyte derived macrophages (MDMs), while inactivation of purinergic receptor P2X7 (with oxidized ATP (OXATP), or NLRP3 (with specific short hairpin RNAs) reduced IL-1β release (FIG. 6, E and F). Conversely, activation of P2Y2 with Diquafosol (a specific P2Y2 agonist used for the treatment of dry eyes (33)), impaired IL-1β release after stimulation with LPS+ATP (FIG. 6I).

Taken together, these results indicate that P2Y2 inhibits NLRP3 inflammasome dependent IL-1β release. Since IL-1β release is a hallmark of pro-inflammatory macrophage activation, we next analyzed the impact of P2Y2 inactivation on functional reprogramming of macrophages (34). P2Y2 inhibition by kaempferol (FIG. 6J-6N and 6Q) or P2Y2 depletion (FIGS. 6O and 10C) promoted pro-inflammatory activation of PMA-treated THP1 macrophages (FIG. 6J) and MDMs (FIG. 6K and 6L-6O), as revealed by the reduced secretion of the immunosuppressive cytokine interleukin-10 (IL-10) (FIG. 6, J and K), the diminished membrane expression of anti-inflammatory macrophage activation marker CD163 (FIG. 6, L and M), the increased expression of the pro-inflammatory transcription factor IFN regulatory factor 5 (IRF5) (35) (FIG. 6, N and O) and the induction of the majority (90%) of known human markers of macrophage activation (FIG. 6Q). Many of these human markers (including CCL18 (36), IL-8 (37, 38), CCL20 (38), SOD2 (36), TNF (9, 37, 38) and IL-1β (9, 38)) have been found increased during severe COVID-19 disease. P2Y2 inactivation or depletion induced pro-inflammatory macrophage activation to the same extent as the positive control, interferon-γ (IFNγ (FIG. 6, G and N). Moreover, Diquafosot impaired IFNγ-mediated increase expression of IFN regulatory factor 5 (IRF5) (FIG. 6P), thus demonstrating that P2Y2 activation represses the pro-inflammatory activation of macrophages. Interestingly, the depletion of NLRP3 increased P2Y2-dependent migration of macrophages induced by LPS+ATP (FIG. 11A). Accordingly, NLRP3 depletion increased F-actin cytoskeletal remodeling (FIG. 11B-D), a process that depends on PYK2Y402* and contributes to macrophage migration after the stimulation with LPS+ATP (39).

Altogether, these results indicate that P2Y2 acts as an endogenous negative modulator of macrophage pro-inflammatory functions and raise the possibility that P2Y2 agonists could be used as candidate drugs for the treatment of COVID-19-associated hyper-inflammation.

### 2.3.2.5. The P2Y2-NLRP3 Interaction Controls the Susceptibility to SARS COV2 Through the Modulation of Viral Entry.

Since the P2Y2-NLRP3 interaction also increased in A549 type II pneumocytes ((FIG. 4, I and J) that were infected with SARS-CoV-2, we then determined whether the P2Y2-NLRP3 interaction may control susceptibility to SARS-CoV-2 as we previously described for HIV-1 (20). We first analyzed expression levels of P2Y2 and NLRP3 during the infection of permissive host cells. The level of purinergic receptor P2Y2 increased after 1 hour and 48 hours of in vitro infection with SARS-CoV-2 of Vero E6 cells (FIG. 7, A) and ACE2-overexpressing A549 (ACE2-A549) cells (FIG. 7, B), respectively. Surprisingly, we detected that purinergic receptor P2X7 is also increased 1 hour post-infection (FIG. 7, C). These processes occur before (FIG. 7, A and C) or at the same time (FIG. 7, B) as the intracellular expression of Spike protein, thus indicating that P2Y2 and P2X7 may contribute to the early steps of SARS-CoV-2 infection. Contrary to what was observed during the early steps of HIV-1 infection (20), SARS-CoV-2 viral infection occurs independently of NLRP3 expression levels (FIG. 7, A and B). We then determined the impact of purinergic receptors on viral replication. Interestingly, the P2Y2 agonists, UTP, Diquafosol and Denufosot, strongly blocked the replication of SARS-CoV-2 and/or reduced associated cytopathogenic effects, in Vero E6 cells (FIG. 7D, 7E, 7I-7K and 12A and 12B) and ACE2-A549 cells (FIG. 7, F-H), as revealed by the detection of RdRp and E mRNA expression (FIG. 7, D-G), the intracellular expression of Spike protein (FIG. 7, H), the frequency of Spike-positive cells (FIG. 7, I and J), and the cellular viability (FIG. 12), demonstrating that P2Y2 acts as an intrinsic restriction factor for SARS-CoV-2 infection.

We next examined the impact of a non-selective antagonist of purinergic receptors P2X, the pyridoxal-phosphate-6-azopheny-2′,4′-disulfonate (PPADS) and OxATP, on SARS-COV-2 viral replication and related cellular damage, to evaluate the possibility that P2X7 might also affect viral replication. The inhibition of P2X and P2X7 activities by PPADS and OxATP reduced the frequency of Spike-positive cells (FIG. 7I, 7J and 13A), intracellular expression of Spike (FIG. 7K) and the cytopathogenic effects (FIG. 13B) elicited by SARS-CoV-2 in Vera E6 cells. PPADS and OxATP partially affected viability of uninfected Vera E6 cells (FIG. 13, C and D) and ACE2-A549 cells (FIG. 13, E and F) at the concentration of 100 μM. Conversely, activation of P2X7 with the 2′(3′)-O-(4-Benzoylbenzoyl) adenosine 5′-triphosphate (BzATP) increased replication of SARS-CoV-2, as revealed by the increase of intracellular Spike expression levels (FIG. 7K), but did not show a significant impact on cytopathogenic effects (FIG. 13G) and cellular viability (FIG. 13, H and I). These results indicate that purinergic receptors P2Y2 and P2X7 regulate the replication of SARS-CoV-2.

We then examined whether purinergic receptors control SARS-CoV-2 replication through the modulation of viral entry. Since ACE2 down-regulation detected during the early steps of SARS-CoV-2 infection follows viral entry (40), Vero E6 cells were infected during 24 hours in presence of modulators of purinergic receptors and assessed for ACE2 expression. P2Y2 agonists (UTP and Diquafosol) and P2X7 antagonist (OxATP) repressed ACE2 down regulation detected in response to SARS-CoV-2 infection as a consequence of viral entry (FIG. 7K). Consistently, BzATP reduces ACE2 expression levels (FIG. 7K), revealing that the purinergic receptors P2Y2 and P2X7 regulate SARS-CoV-2 entry into host cells. To determine whether purinergic receptors may control viral entry through the modulation of ACE2 expression in host cells, Vero E6 (FIG. 14A) and ACE2-A549 cells (FIG. 14B) were incubated during 24 hours in the presence of purinergic receptor modulators and ACE2 membrane expression was analyzed by flow cytometry. The expression of ACE2 did not change in the presence of the agonists of P2Y2 (UTP, Diquafosol and Denufosol) and P2X7 (BzATP), the non-selective antagonist of purinergic receptors P2X (PPADS) and the P2X7 antagonist (OxATP), implying that the purinergic: receptors P2Y2 and P2X7 control the entry of SARS-CoV-2 into host cells without affecting the membrane expression of ACE2 (FIG. 14, A and B), These results also showed that BzATP significantly increased the entry of SARS-CoV2 into host cells (FIG. 7K), without affecting ACE2 membrane expression, thus revealing that during hyper-inflammation, the extracellular accumulation of the physiological agonist of P2X7, ATP could enhance the entry of SARS-CoV-2 into permissive cells and favor viral propagation. Altogether these results indicate that P2Y2 and P2X7 regulate susceptibility to SARS-CoV-2 through the modulation of viral entry.

### 2.3.3. Conclusions

With the goal of studying the potential contribution of the interaction between P2Y2 and NLRP3 to the pathogenesis of SARS-CoV-2 infection, our study revealed that two major sensors of danger signals, namely, P2Y2 and NLRP3, increased their interaction during COVID-19. The enhanced P2Y2-NLRP3 interaction is detected on immune cells, namely neutrophils, monocytes/macrophages and lymphocytes, whose differentiation or specific functions were found to be altered during COVID-19, contributing to organ dysfunction and disease severity. In the majority of these processes, dysregulated immune functions can be regulated by NLRP3. NLRP3-dependent inflammasome activation and uncontrolled extracellular traps (NET) production by neutrophils have been proposed to contribute to hyper-inflammation and dysregulated coagulation in COVID-19 patients with severe disease (41, 42). Activation of the NLRP3 inflammasome and pyroptosis have been detected during SARS-CoV-2 infection (14) and COVID-19 (43). In addition, danger signals such as calprotectin recently associated with COVID-19 disease severity (44), are known to activate the NLRP3 inflammasome (45) or to be released as a consequence of its activation (46) and may contribute to pyroptosis (46). Recent studies showed that numerous patients with severe COVID-19 exhibit a higher plasma concentration of inflammasome-induced cytokines (such as IL-1β and IL-18) than patients with moderate disease (9, 47). Our study reveals that the detection of the P2Y2-NLRP3 interaction positively correlated l with disease severity and increased with viral infection. Accordingly, the detection of P2Y2-NLRP3 interaction on circulating blood cells could be a prognostic marker for the transition between moderate to severe disease during COVID-19.

By deciphering the unexpected relationship between P2Y2 and NLRP3 during COVID-19, our study showed that P2Y2 regulates macrophage functions and represents an endogenous repressor of macrophage pro-inflammatory functions through the negative modulation of NLRP3 inflammasome activation, pro-inflammatory reprogramming and pyroptosis. Our study also demonstrated that purinergic receptors P2Y2 and P2X7 control the susceptibility to SARS-CoV-2. P2Y2 acts as a restriction factor while P2X7 promotes viral entry, without interfering with ACE2 membrane expression. Since we previously observed that the purinergic receptors P2Y2 and P2X7 control HIV-1 envelope-elicited fusion ((20, 22), it is conceivable that P2Y2 and P2X7 may modulate the entry of SARS-CoV-2 into host cells through modulation of the Spike S fusogenic activity. Interestingly, the FDA-approved drug Ivermectin, which is known to potentiate the activity of the purinergic receptor P2X4 (48), which can form heterotrimeric receptors with P2X7 (49), was recently shown to inhibit the replication of SARS-CoV-2 in vitro (50).

According to a recent retrospective study revealing that the early administration of IL-1 receptor antagonist anakinra significantly improved prognosis of COVID-19 patients with severe inflammatory failure (51), our work confirms that targeting inflammasome-dependent IL-1β/IL-18 and/or purinergic signaling pathways may offer a novel opportunity for the treatment of viral infection and hyper-inflammation associated with COVID-19. Our study provides the first rationale for testing P2Y2 agonists such as Diquafosol and Denufosol which have been previously approved or proposed for the treatments of dry eye (33) and cystic fibrosis (52), respectively, alone or in combination with specific antagonists for P2X7 such as CE-224,535 or JNJ-54175446, which were assessed without success for the treatment of rheumatoid arthritis (NCT00628095) or under evaluation as antidepressant for the treatment of major depressive disorders (NCT04116606), respectively. Repurposing of these existing drugs for blocking both viral replication and COVID-19-associated hyper-inflammation should rapidly improve the health of COVID-19 patients.

2.3.4. Validation data

As mentioned above, ATP is able to enhance the entry of SARS-CoV-2 into permissive cells and favor viral propagation. To confirm this, the present inventors used modulators that are able to reduce the level of circulating ATP. They show in FIG. 15 that soluble Apyrase (FIG. 15A, 15C) and the pannexin-1 inhibitor Probenicid (FIG. 15B, 15D), both capable of blocking the ATP release in the extracellular medium, can reduce the susceptibility of ACE2-A549 cells to SARS-COV-2 infection.

Moreover, to confirm that the NLRP3 inflammasome represent a novel molecular target for the treatment of COVID-19, the present inventors used antagonists of NRLP3, as Tranilast, to reduce the inflammasome activity. They demonstrated that the NLRP3 inhibitor Tranilast indeed inhibits SARS-COV-2 replication (FIG. 16).

Finally, to confirm the role of the P2Y2R agonists in the COVID-19-associated hyper-inflammation, the present inventors demonstrated that the P2Y2 agonist Diquafosol abolishes Bleomycin-induced lung inflammation (FIG. 17).

### Bibliographic References

1. W. H. O. R. E. A. f. C.-T. W. Group et al., Association Between Administration of Systemic Corticosteroids and Mortality Among Critically Ill Patients With COVID-19: A Meta-analysis. JAMA, (2020).
2. M. Hoffmann et al., SARS-CoV-2 Cell Entry Depends on ACE2 and TMPRSS2 and Is Blocked by a Clinically Proven Protease Inhibitor. Cell 181, 271-280 e278 (2020).
3. R. Zang et al., TMPRSS2 and TMPRSS4 promote SARS-CoV-2 infection of human small intestinal enterocytes. Sci Immunol 5, (2020).
4. S. Matsuyama et al., Enhanced isolation of SARS-CoV-2 by TMPRSS2-expressing cells. Proc Natl Acad Sci USA 117, 7001-7003 (2020).
5. H. S. Hillen et al., Structure of replicating SARS-CoV-2 polymerase. Nature 58-1, 154-156 (2020).
6. E. de Wit, N. van Doremalen, D. Falzarano, V. J. Munster, SARS and MERS: recent insights into emerging coronaviruses. Nat Rev Microbiol 14, 523-534 (2016).
7. M. Bouhaddou et al., The Global Phosphorylation Landscape of SARS-Cov-2 Infection. Cell 182, 685-712 e619 (2020).
8. X. Cao, COVID-19: immunopathology and its implications for therapy. Nat Rev Immunol 20, 269-270 (2020).
9. C. Lucas et al., Longitudinal analyses reveal immunological misfiring in severe COVID-19. Nature 584, 463-469 (2020).
10. Y. Han et al., Lactate dehydrogenase, an independent risk factor of severe COVID-19 patients: a retrospective and observational study. Aging (Albany NY) 12, 11245-11258 (2020).
11. S. L. Cassel, F. S. Sutterwala, Sterile inflammatory responses mediated by the NLRP3 inflammasome. Eur J Immunol 40, 607-611 (2010).
12. K. V. Swanson, M. Deng, J. P. Ting, The NLRP3 inflammasome: molecular activation and regulation to therapeutics. Nat Rev Immunol 19, 477-489 (2019).
13. I. Jorgensen, M. Rayamajhi, E. A. Miao, Programmed cell death as a defence against infection. Nat Rev Immunol 17, 151-164 (2017).
14. M. Z. Ratajczak et al., SARS-Coy'-2 Entry Receptor ACE2 Is Expressed on Very Small CD45(−) Precursors of Hematopoietic and Endothelial Cells and in Response to Virus Spike Protein Activates the Nlrp3 Inflammasome. Stem Cell Rev Rep, (2020).
15. C. S. Shi, N. R. Nabar, N. N. Huang, J. H. Kehrl, SARS-coronavirus Open Reading Frame-8b triggers intracellular stress pathways and activates NLRP3 inflammasomes. Cell Death Discov 5, 101 (2019).
16. I. Y. Chen, M. Moriyama, M. F. Chang, T. Ichinohe, Severe Acute Respiratory Syndrome coronavirus Viroporin 3a Activates the NLRP3 Inflammasome. Front Microbiol 10, 50 (2019).
17. C. Cekic, J. Linden, Purinergic regulation of the immune system. Nat Rev Immunol 16, 177-192 (2016).
18. G. Burnstock, G. E. Knight, Cellular distribution and functions of P2 receptor subtypes in different systems. Int Rev Cytol 240, 31-304 (2004).
19. V. He, L. Franchi, G. Nunez, TLR agonists stimulate Nlrp3-dependent IL-1 beta production independently of the purinergic P2X7 receptor in dendritic cells and in vivo. J Immunol 190, 334-339 (2013).
20. A. Paoletti et al., HIV-1 Envelope Overcomes NLRP3-Mediated Inhibition of F-Actin Polymerization for Viral Entry. Cell Rep 28, 3381-3394 e3387 (2019).
21. F. Di Virgilio, D. Dal Ben, A. C. Sarti, A. L. Giuliani, S. Falzoni, The P2X7 Receptor in Infection and Inflammation. Immunity 47, 15-31 (2017).
22. C. Seror et al., Extracellular ATP acts on P2Y2 purinergic receptors to facilitate HIV-1 infection, J Exp Med 208, 1823-1834 (2011).
23. D. Ferrari, M. Idzko, T. Muller, R. Manservigi, P. Marconi, Purinergic Signaling: A New Pharmacological Target Against Viruses? Trends Pharmacol Sci 39, 926-936 (2018).
24. L. Carsana et al., Pulmonary post-mortem findings in a series of COVID-19 cases from northern Italy: a two-centre descriptive study. Lancet Infect Dis, (2020).
25. S. A. Rooney, Regulation of surfactant secretion. Comp Biochem Physiol A Mol Integr Physiol 129, 233-243 (2001).
26. D. Myrtek et al., Activation of human alveolar macrophages via P2 receptors: coupling to intracellular Ca2+ increases and cytokine secretion. J Immunol 181, 2181-2188 (2008).
27. H. Guo, J. B. Callaway, J. P. Ting, Inflammasomes: mechanism of action, role in disease, and therapeutics. Nat Med 21, 677-687 (2015).
28. M. R. Elliott et al., Nucleotides released by apoptotic cells act as a find-me signal to promote phagocytic clearance. Nature 461, 282-286 (2009).
29. M. Kronlage et al., Autocrine purinergic receptor signaling is essential for macrophage chemotaxis. Sc; Signal 3, ra55 (2010).
30. M. Merad, J. C. Martin, Pathological inflammation in patients with COVID-19: a key role for monocytes and macrophages. Nat Rev Immunol 20, 355-362 (2020).
31. M. Liao et al., Single-cell landscape of bronchoalveolar immune cells in patients with COVID-19. Nat Med 26, 842-844 (2020).
32. Q. Wu et al., NOX2-dependent ATM kinase activation dictates pro-inflammatory macrophage phenotype and improves effectiveness to radiation therapy. Cell Death Differ 24, 1632-1644 (2017).
33. G. M. Keating, Diquafosol ophthalmic solution 3%: a review of its use in dry eye. Drugs 75, 911-922 (2015).
34. P. J. Murray et al., Macrophage activation and polarization: nomenclature and experimental guidelines. Immunity 41, 14-20 (2014).
35. T. Krausgruber et al., IRF5 promotes inflammatory macrophage polarization and TH1-TH17 responses. Nat Immunol 12, 231-238 (2011).
36. P. Bost et al., Host-Viral Infection Maps Reveal Signatures of Severe COVID-19 Patients. Cell 181, 1475-1488 e1412 (2020).
37. D. M. Del Valle et al., An inflammatory cytokine signature predicts COVID-19 severity and survival. Nat Med, (2020).
38. R. L. Chua et al., COVID-19 severity correlates with airway epithelium-immune cell interactions identified by single-cell analysis. Nat Biotechnol 38, 970-979 (2020).
39. M. Okigaki et al., Pyk2 regulates multiple signaling events crucial for macrophage morphology and migration. Proc Nati Acad Sci USA 100, 1 0740-1 0745 (2003).
40. I. Glowacka et al., Differential downregulation of ACE2 by the spike proteins of severe acute respiratory syndrome coronavirus and human coronavirus NL63. J Virol 84, 1198-1205 (2010).
41. M. Karmakar, M. A. Katsnetson, G. R. Dubyak, C. Pearlman, Neutrophil P2X7 receptors mediate NLRP3 inflammasome-dependent IL-1beta secretion in response to ATP. Nat Commun 7, 10555 (2016).
42. M. Leppkes et al., Vascular occlusion by neutrophil extracellular traps in COVID-19. EBioMedicine 58, 102925 (2020).
43. A. Kroemer et al., Inflammasome activation and pyroptosis in tymphopenic liver patients with COVID-19. J Hepatol, (2020).
44. A. Silvin et al., Elevated Calprotectin and Abnormal Myeloid Cell Subsets Discriminate Severe from Mild COVID-19. Cell, (2020).
45. J. C. Simard et al., S100A8 and S100A9 induce cytokine expression and regulate the NLRP3 inflammasome via ROS-dependent activation of NF-kappaB(1.). PLoS One 8, e72138 (2013).
46. A. A. Basiorka et al., The NLRP3 inflammasome functions as a driver of the myelodysplastic syndrome phenotype. Blood 128, 2960-2975 (2016).
47. E. J. Giamarellos-Bourboulis et al., Complex Immune Dysregulation in COVID-19 Patients with Severe Respiratory Failure. Cell Host Microbe 27, 992-1000 e1003 (2020).
48. L. Stokes, J. A. Layhadi, L. Bibic, K. Dhuna, S. J. Fountain, P2X4 Receptor Function in the Nervous System and Current Breakthroughs in Pharmacology. Front Pharmacol 8, 291 (2017).
49. C. Guo, M. Masin, O. S. Qureshi, R. D. Murrell-Lagnado, Evidence for functional P2X4/P2X7 heteromeric receptors. Mol Pharmacol 72, 1447-1456 (2007).
50. L. Caly, J. D. Druce, M. G. Catton, D. A. Jans, K. M. Wagstaff, The FDA-approved drug ivermectin inhibits the replication of SARS-CoV-2 in vitro. Antiviral Res 178, 104787 (2020).
51. R. Cauchois et al., Early IL-1 receptor blockade in severe inflammatory respiratory failure complicating COVID-19. Proc Natl Acad Sci U S A 117, 18951-18953 (2020).
52. F. Ratjen et al., Long term effects of denufesol tetrasodium in patients with cystic fibrosis. J Cyst Fibros 11, 539-549 (2012).
53. D. E. Gordon et al., A SARS-CoV-2 protein interaction map reveals targets for drug repurposing. Nature 583, 459-468 (2020).
54. P. Maisonnasse et al., Hydroxychloroquine use against SARS-CoV-2 infection in non-human primates. Nature, (2020).
55. A. Allouch et al., p21-mediated RNR2 repression restricts HIV-1 replication in macrophages by inhibiting dNTP biosynthesis pathway. Proc Nati Acad Sci USA 110, E3997-4006 (2013).
56. A. Allouch et al., SUGT1 controls susceptibility to HIV-1 infection by stabilizing microtubule plus-ends. Cell Death Differ, (2020).
57. B. Hanley, S. B. Lucas, E. Youd, B. Swift, M. Osborn, Autopsy in suspected COVID-19 cases. J Clin Pathol 73, 239-242 (2020).
58. L. Homolya, W. C. Watt, E. R. Lazarowski, B. H. Koller, R. C. Boucher, Nucleotide-regulated calcium signaling in lung fibroblasts and epithelial cells from normal and P2Y(2) receptor (−/−) mice. J Biol Chem 274, 26454-26460 (1999).
59. L. Meziani et al., CSF1R inhibition prevents radiation pulmonary fibrosis by depletion of interstitial macrophages. Eur Respir J 51, (2018).
60. L. Falasca et al., Post-Mortem Findings in Italian Patients with COVID-19—a Descriptive Full Autopsy Study of cases with and without co-morbidities. J Infect Dis, (2020).
Abdi M H, et al. Discovery and structure-activity relationships of a series of pyroglutamic acid amide antagonists of the P2X7 receptor. Bioorg Med Chem Lett. 2010 Sep. 1 ;20(17):5080-4.
Agostini L, et al. NALP3 forms an IL-1 beta-processing inflammasome with increased activity in Muckle-Wells autoinflammatory disorder. Immunity. 2004 March; 20(3):319-25.
Bhattacharya A, et al. Pharmacological characterization of a novel centrally permeable P2X7 receptor antagonist: JNJ-47965567. Br J Pharmacal. 2013 October; 170(3):624-40.
Broom D C, et al. Characterization of N-(adamantan-1-ylmethyl)-5-[(3R-amino-pyrrolidin-1-yl)methyl]-2-chloro-benzamide, a P2X7 antagonist in animal models of pain and inflammation. J Pharmacol Exp Ther. 2008 December; 327(3):620-33.
Brummelkamp T R, et al. A system for stable expression of short interfering RNAs in mammalian cells. Science. 2002 Apr. 19; 296(5567):550-3.
Camell C, et al. Regulation of Nlrp3 inflammasome by dietary metabolites. Semin Immunol. 2015 September; 27(5):334-42.
Carmo M R, et al. The P2X7 receptor antagonist Brilliant Blue G attenuates contralateral rotations in a rat model of Parkinsonism through a combined control of synaptotoxicity, neurotoxicity and gliosis. Neuropharmacology. 2014 June; 81:142-52.
Coll R C, et al. A small-molecule inhibitor of the NLRP3 inflammasome for the treatment of inflammatory diseases. Nat Med. 2015 March: 21(3):248-55.
Dong E, et al. An interactive web-based dashboard to track COVID-19 in real time. Lancet Infect Dis. 2020 May; 20(5):533-534.

Donnelly-Roberts D L and Jarvis M F. Discovery of P2X7 receptor-selective antagonists offers new insights into P2X7 receptor function and indicates a rote in chronic pain states. Br J Pharmacol. 2007 July; 151(51:571-9.

Duffy E B, et al. FcgammaR mediates TLR2- and Syk-dependent NLRP3 inflammasome activation by inactivated Francisetta tularensis LVS immune complexes. J Leukoc Biot. 2016 December; 100(6):1335-1347.

Duplantier A J, et al. Optimization of the physicochemical and pharmacokinetic attributes in a 6-azauracil series of P2X7 receptor antagonists leading to the discovery of the clinical candidate CE-224,535. Bioorg Med Chem Lett. 2011 Jun. 15; 21(12):3708-11.

Elbashir S M, et al. Duplexes of 21-nucteotide RNAs mediate RNA interference in cultured mammalian cells. Nature. 2001 May 24; 411(6836):494-8.

Fan E, et al. Acute Respiratory Distress Syndrome: Advances in Diagnosis and Treatment. JAMA. 2018 Feb. 20; 319(7):698-710.

Fanelli V and Ranieri V M. Mechanisms and clinical consequences of acute lung injury. Ann Am Thorac Soc. 2015 March; 12 Suppl 1:S3-8.

Gargett C E and, Wiley J S. The isoquinoline derivative KN-62 a potent antagonist of the P2Z-receptor of human lymphocytes. Br J Pharmacot. 1997 April; 120(8):1483-90.

Guan B, et al. Sensitive extraction-free SARS-CoV-2 RNA virus detection using a novel RNA preparation method. medRxiv. 2021 Feb. 1: 2021.01.29.21250790.

Guan W J and Zhong N S. Clinical Characteristics of Covid-19 in China. Reply. N Engl J Med. 2020 May 7; 382(19)1861-1862.

Guile S D, et al. Antagonists of the P2X(7) receptor. From lead identification to drug development. J Med Chem. 2009 May 28; 52(10):3123-41.

Halle A, et al. The NALP3 inflammasome is involved in the innate immune response to amytoid-beta. Nat Immunol. 2008 August: 9(8):857-65.

Hannon G J. RNA interference. Nature. 2002 Jul. 11: 418(6894):244-51.

Herold 5, et al. Acute lung injury: how macrophages orchestrate resolution of inflammation and tissue repair. Front Immunol. 2011 Nov. 24; 2:65.

Hillmann P, et al. Key determinants of nucleotide-activated G protein-coupled P2Y(2) receptor function revealed by chemical and pharmacological experiments, mutagenesis and homology modeling. J Med Chem. 2009 May 14; 52(9):2762-75.

Homerin G, et al. Pyrogtutamide-Based P2X7 Receptor Antagonists Targeting Inflammatory Bowel Disease. J Med Chem. 2020 Mar. 12; 63(5):2074-2094.

Honore P, et al. A-740003 [N-(1-{[(cyanoimino)(5-quinolinytamino) methyt]amino}-2,2-dimethylpropyl)-2-(3,4-dimethoxyphenyl)acetamide], a novel and selective P2X7 receptor antagonist, dose-dependently reduces neuropathic pain in the rat. J Pharmacol Exp Ther. 2006 December; 319(3):1376-85.

Huang C, et al. Clinical features of patients infected with 2019 novel coronavirus in Wuhan, China. Lancet. 2020 Feb. 15; 395(10223):497-506.

Huang Y, et al. Tranilast directly targets NLRP3 to treat inflammasome-driven diseases. EMBO Mol Med. 2018 April; 10(4):e8689.

Jacobson K A, et al. Development of selective agonists and antagonists of P2Y receptors. Purinergic Signal. 2009 March; 5(1):75-89.

Jacobson K A, et al. G protein-coupled adenosine (P1) and P2Y receptors: ligand design and receptor interactions. Purinergic Signal. 2012 September; 8(3):419-36.

Kawate T, et al. Crystal structure of the ATP-gated P2X(4) ion channel in the closed state. Nature. 2009 Jul, 30; 460(7255):592-8.

Kellerman D, et at. Inhaled P2Y2 receptor agonists as a treatment for patients with Cystic Fibrosis lung disease. Adv Drug Deliv Rev. 2002 Dec. 5; 54(11):1463-74.

Khalafalla M G, et al. P2X7 receptor antagonism prevents IL-1β release from salivary epithelial cells and reduces inflammation in a mouse model of autoimmune execrinopathy. J Biol Chem. 2017 Oct. 6; 292(40):16626-16637.

Kriegler M. Assembly of enhancers, promoters, and splice signals to control expression of transferred genes Methods Enzymol. 1990; 185:512-27.

Lamkanfi M, et al. Glyburide inhibits the Cryopyrin/Nalp3 inflammasome. J Cell Biol. 2009 Oct. 5; 187(1):61-70.

Liu L, et al. Anti-spike IgG causes severe acute lung injury by skewing macrophage responses during acute SARS-CoV infection. JCI Insight. 2019 Feb. 21; 4(4):e123158.

Lowe P N and Beechey R B. Interactions between the mitochondrial adenosinetriphosphatase and periodate-oxidized adenosine 5′-triphosphate, an affinity label for adenosine 5′-triphosphate binding sites. Biochemistry 1982, 21, 17, 4073-4082

Luyt CÉ, et al. Virus-induced acute respiratory distress syndrome: epidemiology, management and outcome. Presse Med, 2011 December; 40(12 Pt 2):e561-8.

McInnes I B, et al. Targeting the P2X7 receptor in rheumatoid arthritis: biological rationale for P2X7 antagonism. Clin Exp Rheumatol. 2014 November-December; 32(6):878-82.

McManus M T and Sharp P A. Gene silencing in mammals by small interfering RNAs. Nat Rev Genet. 2002 October; 3(10):737-47.

Michel A D, et al, Negative and positive allosteric modulators of the P2X(7) receptor. Br J Pharmacol, 2008 February; 153(4):737-50.

Moeckel D, et al. Optimizing human apyrase to treat arterial thrombosis and limit reperfusion injury without increasing bleeding risk. Sci Transl Med, 2014 Aug. 6; 6(248):248ra105.

Murry M A and Wolk C P. Identification and initial utilization of a portion of the smaller plasmid of Anabaena variabilis ATCC 29413 capable of replication in Anabaena sp. strain M-131. Mol Gen Genet. 1991 May; 227(1):113-9.

North R A. Molecular physiology of P2X receptors. Physiol Rev. 2002 October; 82(4):1013-67.

Shao B Z, et al. NLRP3 inflammasome and its inhibitors: a review. Front Pharmacol. 2015 Nov. 5; 6:262.

Sun C, et al. Protective effect of different flavonoids against endothelial senescence via NLRP3 inflammasome. J Functional Foods. Volume 26, October 2016, Pages 598-609

Thevarajan I, et al. Clinical presentation and management of COVID-19. Med J Aust. 2020 August; 213(3):134-139.

Tulapurkar M E, et al. Endocytosis mechanism of P2Y2 nucleotide receptor tagged with green fluorescent protein: clathrin and actin cytoskeleton dependence. Cell Mol Life Sci. 2005 June; 62(12):1388-99.

Tuschl T, et al. Targeted mRNA degradation by double-stranded RNA in vitro. Genes Dev. 1999 Dec. 15; 13(24):3191-7.

Vandanmagsar 8, et al. The NLRP3 inflammasome instigates obesity-induced inflammation and insulin resistance. Nat Med. 2011 February; 17(2):179-88.

Van Kolen K, et al. P2Y12 receptor signalling towards PKB proceeds through IGF-I receptor cross-talk and requires activation of Src, Pyk2 and Rap1. Cell Signal. 2006 August; 18(8):1169-81.

Van Poecke S, et al. Synthesis and P2Y₂ receptor agonist activities of uridine 5′-phosphonate analogues. Bioorg Med Chem. 2012 Apr. 1; 20(7):2304-15.

Wang D, et al. Clinical Characteristics of 138 Hospitalized Patients With 2019 Novel coronavirus-Infected Pneumonia in Wuhan, China. JAMA. 2020 Mar. 17; 323(11):1061-1069.

Ware L B and Matthay M A. The acute respiratory distress syndrome. N Engl J Med. 2000 May 4; 342(18):1334-49.

Yao X, et al. In Vitro Antiviral Activity and Projection of Optimized Dosing Design of Hydroxychloroquine for the Treatment of Severe Acute Respiratory Syndrome coronavirus 2 (SARS-CoV-2). Clin Infect Dis. 2020 Jul. 28; 71(15):732-739.

Youm Y H, et al. The ketone metabolite β-hydroxybutyrate blocks NLRP3 inflammasome-mediated inflammatory disease. Nat Med. 2015 March; 21(3):263-9.

Zhou Y, et al. Aberrant pathogenic GM-CSF+ T cells and inflammatory CD14+CD16+ monocytes in severe pulmonary syndrome patients of a new coronavirus. bioRx v 2020.02.12.945576;

1.-25. (canceled)

26. Method for treating a subject suffering from an inflammatory disease associated with an accumulation of pro-inflammatory macrophages and/or associated with an over-activation of the NRLP3 inflammasome, said method comprising the step of administering to said subject an agonist of a purinergic P2Y receptor.

27. The method of claim 26, wherein said inflammatory disease is Acute Respiratory Distress Syndrome (ARDS), the cryopyrin associated periodic syndrome, rheumatoid arthritis, obesity, or Alzheimer's disease.

28. The method of claim 26, wherein said inflammatory disease is an ARDS caused by sepsis, pneumonia, pancreatitis, surgery, radiation, virus, or a chemotherapeutic drug.

29. The method of claim 26, wherein said inflammatory disease is a virus-induced ARDS.

30. The method of claim 26, wherein said inflammatory disease is the COVID19 disease associated with the infection of the Severe Acute Respiratory Syndrome coronavirus 2.

31. The method of claim 26, wherein said agonist is an agonist of the P2Y2 receptor.

32. The method of claim 26, wherein said agonist is MRS2698, Uridine triphosphate (UTP), 4-thio-UTP, 2-thioUTP, Diquafosol, PSB1114, ATP, Denufosol, Ap4A, UTPγS, 5BrUTP, or MRS2768.

33. The method of claim 26, wherein said agonist is used to impair the replication of the Severe Acute Respiratory Syndrome coronavirus 2 in epithelial cells and for treating a subject suffering from the COVID19 disease.

34. The method of claim 26, wherein said agonist is administered to a subject suffering from an ARDS caused by an influenza virus, a respiratory virus, or an herpesvirus.

35. The method of claim 26, wherein said agonist is administered to a subject suffering from a coronavirus.

36. The method of claim 26, wherein said agonist is administered to a subject suffering from a Betacoronavirus.

37. The method of claim 26, wherein said agonist is administered to a subject suffering from a SARS-CoV-2 virus.

38. A method for impairing the replication of the Severe Acute Respiratory Syndrome coronavirus 2 in epithelial cells, said method comprising the step of contacting said cells with an agonist of a purinergic P2Y receptor.

39. The method of claim 38, wherein said agonist is an agonist of the P2Y2 receptor.

40. The method of claim 38, wherein said agonist is MRS2698, Uridine triphosphate (UTP), 4-thio-UTP, 2-thioUTP, Diquafosol, PSB1114, ATP, Denufosol, Ap4A, UTPγS, 5BrUTP, or MRS2768.

41. The method of claim 38, wherein said agonist is used to impair the replication of the Severe Acute Respiratory Syndrome coronavirus 2 in epithelial cells and for treating a subject suffering from the COVID19 disease.

42. The method of claim 38, wherein said agonist is administered to a subject suffering from an ARDS caused by an influenza virus, a respiratory virus, or an herpesvirus.

43. The method of claim 38, wherein said agonist is administered to a subject suffering from a coronavirus.

44. The method of claim 38, wherein said agonist is administered to a subject suffering from a Betacoronavirus.

45. The method of claim 38, wherein said agonist is administered to a subject suffering from a SARS-CoV-2 virus.
